(19) United States
(12) Patent Application Publication
(10) Pub. No.: US 2023/0158170 A1
(43) Pub. Date: May 25, 2023

(54) IL-1Ra GENE THERAPY FOR INTERVERTEBRAL DISC DEGENERATION

(71) Applicant: Pacira Therapeutics, Inc., Tampa, FL (US)

(72) Inventors: Rebecca K. SENTER, Arlington, MA (US); Christine L. LE MAITRE, Derbyshire (GB); Joseph W. SNUGGS, Sheffield (GB)

(21) Appl. No.: 17/821,761

(22) Filed: Aug. 23, 2022

Related U.S. Application Data
(60) Provisional application No. 63/236,580, filed on Aug. 24, 2021.

(51) Int. Cl. A61K 48/00; C12N 15/861; A61P 19/02; C07K 14/545

(52) U.S. Cl. CPC A61K 48/005; A61K 48/0075; C12N 15/861; A61P 19/02; C07K 14/545; C12N 2750/14143

(57) ABSTRACT

The disclosure relates to pharmaceutical compositions comprising an adenoviral-based biological delivery and expression system encoding human or mammalian interleukin-1 receptor antagonist (IL-1Ra) and methods of using the pharmaceutical compositions for expressing IL-IRA in cells of one or more intervertebral discs of a subject suffering from degenerative disc disease (DDD) or a condition associated with DDD, and for treatment of DDD or conditions associated with DDD.

### INCORPORATION-BY-REFERENCE OF SEQUENCE LISTING

The instant application contains a Sequence Listing which has been submitted in ASCII format via EFS-Web and is hereby incorporated by reference in its entirety. Said ASCII copy, created on Aug. 23, 2022, is named “PCRTX_017A_Sequence_Listing.xml” and is 97.5 kilobytes in size.

### BACKGROUND OF THE INVENTION

Low back pain affects 80% of the population at some point in their lives, with almost half of cases attributed to intervertebral disc (IVD) degeneration. IVD degeneration is triggered by the native IVD cells themselves; where a cascade of cytokine and catabolic enzyme production leads to the destruction of extracellular matrix (ECM) and altered disc biomechanics, contributing to the loss of IVD function, which combined with induction of nerve ingrowth and production of neurotrophic factors is thought to contribute to back pain. However, to date the majority of treatments focus on short term pain management, physical therapy and surgical removal of herniated tissue, with none targeting the underlying pathophysiological causes of IVD degeneration. Gene therapy approaches present novel and exciting possibilities for the treatment of a plethora of diseases.

### SUMMARY OF THE INVENTION

IL-1 is known to act as a pleiotropic cytokine during disc degeneration and loss of IL-1Ra in a knockout model induced spontaneous disc degeneration. Accordingly, there is a clear and unmet medical need for more efficacious, sustained and cost-effective method for IVD treatment by blocking IL-1 in cells and tissues of IVD. The present disclosure describes compositions comprising an adenoviral delivery and expression system (FX201, humantakinogene hadenovec, also referred to as PCRX-201 herein), and methods of using the same to infect cells of human intervertebral discs, including those in subjects suffering from degenerative disc disease (DDD) or a condition associated with DDD, to increase production and release of IL-1Ra, and methods of treating DDD or a condition associated with DDD.

In some embodiments, the present disclosure provides a pharmaceutical composition for treatment of degenerative disc disease (DDD) or a condition associated with DDD, in a subject in need thereof, which comprises an amount, optionally an effective amount, of an adenoviral-based biological delivery and expression system (an adenoviral-based vector or helper-dependent adenoviral vector) comprising a nucleic acid sequence encoding a human interleukin-1 receptor antagonist (IL-1Ra) protein, left and right inverted terminal repeats, an adenoviral packaging signal and non-viral, and non-coding stuffer nucleic acid sequences, wherein the expression of the human IL-1Ra gene is regulated by a NF-kB inducible promoter, which is located upstream of the reading frame of the nucleic acid sequence encoding the human IL-1Ra protein, and wherein the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 95% homologous or identical to the nucleic acid sequence of SEQ ID NO: 7.

The condition associated with DDD can be lower back pain, decreased back muscle tone, reduced flexibility of the back or blood clot or a combination thereof.

The nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 99% homologous or identical to the nucleic acid sequence of SEQ ID NO: 7.

The nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is identical to the nucleic acid sequence of SEQ ID NO: 7

The IL-1Ra in the nucleic acid sequence of the adenoviral-based biological delivery and expression system can comprise the nucleic acid of SEQ ID NO 4.

The nucleic acid according to SEQ ID NO: 4 can express a human IL-1Ra protein of amino acid sequence that is at least 95% homologous or identical to SEQ ID NO: 6.

In some embodiments, the pharmaceutical composition disclosed herein, for treatment of DDD or a condition associated with DDD, is formulated for delivering the adenoviral-based biological delivery and expression system directly into the cells of one or more intervertebral discs of the subject in need thereof. In some embodiments, the pharmaceutical composition disclosed herein is formulated for delivering the adenoviral-based biological delivery and expression system into cells of the cartilaginous endplates (CEP), the highly organized annulus fibrosus (AF) and the central gelatinous nucleus pulposus (NP) region (nucleus pulposus (NP) cells) or a combination thereof, of the one or more intervertebral discs. The one or more intervertebral discs of the subject in need thereof is degenerate discs or non-degenerate discs or both.

In some embodiments, the present disclosure also provides a method of infecting cells of one or more intervertebral discs of a subject suffering from degenerative disc disease (DDD), with an adenoviral-based biological delivery and expression system, wherein the method comprises the steps of: a) infecting cells of one or more intervertebral discs of the subject in need thereof with the pharmaceutical composition comprising an amount, optionally an effective amount, of an adenoviral-based biological delivery and expression system of the present disclosure; and b) expressing IL-1Ra in the cells of the one or more intervertebral discs.

In some embodiments, the method further comprises step c) monitoring the treatment or progress of DDD in the degenerated intervertebral discs of the subject following the expression of the IL-1Ra in (b). In some embodiments, the method further comprises the steps of: (d) continuing to administer the same effective amount of the adenoviral-based biological delivery and expression system to the cells of the one or more intervertebral discs of (a), if monitoring of (c) shows that the degenerative disc disease in the intervertebral disc of the subject is not managed or treated; or (e) further adjusting the amount of the adenoviral-based biological delivery and expression system and administering to the intervertebral discs of (a), if monitoring of (c) shows that the degenerative disc disease in the intervertebral disc of the subject has progressed.

Any of the above aspects, or aspects otherwise disclosed herein, can be combined with any other aspect.

Embodiments of the disclosure include the following numbered embodiments:

1. A pharmaceutical composition for treatment of degenerative disc disease (DDD) or a condition associated with DDD, in a subject in need thereof, comprising an adenoviral-based biological delivery and expression system comprising a nucleic acid encoding a interleukin-1 receptor antagonist (IL-1Ra) protein.

2. The pharmaceutical composition of embodiment 1, wherein the nucleic acid further comprises left and right inverted terminal repeats, an adenoviral packaging signal and non-viral, and non-coding stuffer nucleic acid sequences.

3. The pharmaceutical composition of embodiment 1 or 2, wherein the nucleic acid further comprises an inflammation-sensitive promoter located upstream of the reading frame of the nucleic acid sequence encoding the IL-1Ra protein, such that expression of the IL-1Ra gene is regulated by the inflammation-sensitive promoter, optionally wherein the inflammation-sensitive promoter is selected from the group consisting of a promoter inducible by NF-KB, interleukin 6 (II-6), interleukin-1 (IL-1), tumor necrosis factor (TNF), cyclooxygenase 2 (COX-2), complement factor 3 (C3), serum amyloid A3 (SAA3), and macrophage inflammatory protein-1a (MIP-1a), or hybrid constructs thereof.

4. The pharmaceutical composition of embodiment 1 or 3, wherein the nucleic acid further comprises an NF-kB inducible promoter located upstream of the reading frame of the nucleic acid sequence encoding the IL-1Ra protein, such that expression of the IL-1Ra gene is regulated by the NF-kB inducible promoter.

5. The pharmaceutical composition of embodiment 1 or 4, wherein the nucleic acid comprises or consists of a nucleic acid sequence that is, or is at least, 80%, 85%, 90%, 95%, 96%, 97%, 98%, 99% or 100% identical to the nucleic acid sequence of SEQ ID NO: 7.

6. The pharmaceutical composition of any one of embodiments 1-5, wherein the condition associated with DDD is lower back pain, decreased back muscle tone, reduced flexibility of the back, blood clot or a combination thereof.

7. The pharmaceutical composition of any one of embodiments 1-6, wherein the sequence of the nucleic acid encoding IL-1Ra comprises or consists of a nucleic acid sequence that is, or is at least, 80%, 85%, 90%, 95%, 96%, 97%, 98%, 99% or 100% identical to the nucleic acid of SEQ ID NO 4.

8. The pharmaceutical composition of any one of embodiments 1-7, wherein the sequence of the nucleic acid encoding IL-1Ra comprises or consists of a nucleic acid sequence that encodes an IL-1Ra protein comprising or consisting of an amino acid sequence that is, or is at least, 80%, 85%, 90%, 95%, 96%, 97%, 98%, 99% or 100% identical to the amino acid sequence of SEQ ID NO: 6.

9. The pharmaceutical composition of any one of embodiments 1-8, wherein the IL-1Ra protein is human IL-1Ra.

10. The pharmaceutical composition of any one of embodiments 1-9, wherein the adenoviral-based biological delivery and expression system further comprises a nucleic acid encoding a protein in addition to interleukin-1 receptor antagonist (IL-1Ra) protein, optionally wherein the additional protein is a therapeutic protein.

11. The pharmaceutical composition of any one of embodiments 1-10, wherein the pharmaceutical composition is formulated for delivering the adenoviral-based biological delivery and expression system directly to the cells of one or more intervertebral discs of the subject in need thereof.

12. The pharmaceutical composition of any one of embodiments 1-11, wherein the one or more intervertebral discs of the subject in need thereof are degenerate discs or non-degenerate discs or both.

13. The pharmaceutical composition of embodiment 12, wherein the one or more intervertebral discs of the subject in need thereof are degenerate discs.

14. The pharmaceutical composition of any one of embodiments 1-13, wherein the pharmaceutical composition is formulated for delivering the adenoviral-based biological delivery and expression system into cells of the cartilaginous endplates (CEP), the highly organized annulus fibrosus (AF) and the central gelatinous nucleus pulposus (NP) region (nucleus pulposus (NP) cells) or a combination thereof, of the one or more intervertebral discs.

15. The pharmaceutical composition of embodiment 14, wherein the pharmaceutical composition is formulated for delivering the adenoviral-based biological delivery and expression system into the nucleus pulposus (NP) cells.

16. The pharmaceutical composition of embodiment 14, wherein the cells are nucleus pulposus (NP) cells.

17. The pharmaceutical composition of any one of embodiments 1-16, wherein only the cells of the degenerate discs infected with the adenoviral-based biological delivery and expression system produce IL-1Ra.

18. The pharmaceutical composition of any one of embodiments 1-17, wherein the cells of one or more intervertebral discs of the subject in need thereof, express IL-1Ra for a period of at least 2 weeks, at least 1 month, at least 3 months, at least 6 months or at least 1 year.

19. The pharmaceutical composition of any one of embodiments 1-18, wherein the pharmaceutical composition comprises an amount of the adenoviral-based biological delivery and expression system effective to treat the degenerative disc disease (DDD) and/or the condition associated with DDD in a subject when administered to the subject.

20. The pharmaceutical composition of any one of embodiments 1-18, wherein the subject does not have Facet Joint Syndrome (FJS).

21. The pharmaceutical composition of any one of embodiments 1-18, wherein the subject has Facet Joint Syndrome (FJS).

22. A method of expressing IL-IRA in cells of one or more intervertebral discs of a subject suffering from degenerative disc disease (DDD) or a condition associated with DDD the method comprising:

a) infecting cells of one or more intervertebral discs of the subject in need thereof with the pharmaceutical composition of any one of embodiments 1-21; and

b) expressing IL-1Ra in the cells of the one or more intervertebral discs.

23. The method of embodiment 22, wherein the adenoviral-based biological delivery and expression system further comprises a nucleic acid encoding a protein in addition to interleukin-1 receptor antagonist (IL-1Ra) protein, optionally wherein the additional protein is a therapeutic protein, and wherein the method further comprises expressing the additional protein in the cells of the one or more intervertebral discs.

24. The method according to embodiment 22 or 23, wherein the cells of the one or more intervertebral discs are infected once with the adenoviral-based biological delivery and expression system.

25. The method according to embodiment 22 or 23, wherein the cells of the one or more intervertebral discs are infected two or more times with the adenoviral-based biological delivery and expression system.

26. The method according to embodiment 25, wherein when the cells of the one or more intervertebral discs are infected two or more times with an adenoviral-based biological delivery and expression system, each infection comprises a different number of genome copies of the adenoviral-based vector.

27. The method according to embodiment 25, wherein, when the cells of the one or more intervertebral discs are infected two or more times with an adenoviral-based biological delivery and expression system, each infection comprises the same number of genome copies of the adenoviral-based vector.

28. The method according to any one of embodiments 25-27, wherein, when the cells of the one or more intervertebral discs are infected two or more times with an adenoviral-based biological delivery and expression system, each infection is done in the same intervertebral disc of the subject.

29. The method according to any one of embodiments 25-27, wherein when the cells of the one or more intervertebral discs are infected two or more times with an adenoviral-based biological delivery and expression system, every second and subsequent infection is done in an intervertebral disc of the subject that is different than the intervertebral disc in which the previous infection was done.

30. The method according to any one of embodiments 22-29, wherein the infecting of the cells of the one or more intervertebral discs comprises injecting the pharmaceutical composition into the cartilaginous endplates (CEP) region, the highly organized annulus fibrosus (AF) region or the central gelatinous nucleus pulposus (NP) region (nucleus pulposus (NP) cells) or a combination thereof, of the one or more intervertebral discs.

31. The method of embodiment 30, wherein the infecting of the cells of the one or more intervertebral discs comprises injecting the pharmaceutical composition into the nucleus pulposus (NP) region of the one or more intervertebral discs.

32. The method of any one of embodiments 22-31, wherein the method treats the degenerative disc disease (DDD) and/or the condition associated with DDD in the subject.

33. The method of any one of embodiments 22-32, further comprises the step of:

c) monitoring the treatment or progress of DDD or the condition associated with DDD in the degenerated intervertebral discs of the subject following the expression of the IL-1Ra in (b).

34. The method of embodiment 33, wherein the monitoring of the treatment or progress of DDD or the condition associated with DDD, is done by determining scores from patient-reported pain and/or function measurements.

35. The method of embodiment 34, wherein determining scores from patient-reported pain is done using visual analog scale (VAS).

36. The method of any one of embodiments 22-35, wherein a VAS score of the subject is lower post-infection of the cells of one or more intervertebral discs of the subject with the pharmaceutical composition.

37. The method of any one of embodiments 34-36, wherein the determining scores from patient-function measurements is done using Oswestry disability index (ODI).

38. The method of any one of embodiments 22-37, wherein a VAS score of the subject is lower post-infection of the cells of one or more intervertebral discs of the subject with the pharmaceutical composition.

39. The method of embodiment 33, wherein the monitoring of the treatment or progress of DDD or the condition associated with DDD comprises determining the level of a marker in the subject selected from the group consisting of: NGF, NT-3, VEGF, Substance P, cytokines, aggrecan, collagen type II, and a combination thereof.

40. The method of embodiment 39, wherein the cytokine is selected from the group consisting of IL-1β, IL-6, IL-8, TNF α, MMP 3, MMP 13, ADAMTS 4, and combinations thereof.

41. The method of any one of embodiments 22-40, wherein one or more of the following occurs:

(a) a decrease or no change in level of one or more of: NGF, NT-3, VEGF, Substance P, IL-1β, IL-6, IL-8, TNF α, MMP 3, MMP 13, or ADAMTS 4, or a combination thereof;

(b) an increase in the level of aggrecan or collagen type II or a combination thereof; or

(c) both,

in the one or more intervertebral discs post-infection of the cells of one or more intervertebral discs of the subject with the pharmaceutical composition.

42. The method of any one of embodiments 22-41, wherein level of aggrecan increases in the one or more intervertebral discs post-infection of the cells of one or more intervertebral discs of the subject with the pharmaceutical composition.

43. The method of embodiment 33, wherein the monitoring of the treatment or progress of DDD or the condition associated with DDD, is done by determining change in a score based on histopathology scoring system for human intervertebral disc degeneration for the one or more intervertebral discs post-infection of the cells of one or more intervertebral discs of the subject with the pharmaceutical composition.

44. The method of any one of embodiments 22-43, wherein a decrease in score based on histopathology scoring system for human intervertebral disc degeneration for the one or more intervertebral discs occurs post-infection of the cells of one or more intervertebral discs of the subject with the pharmaceutical composition.

45. The method of any one of embodiments 33-44, further comprises the steps of:

(d) continuing to administer the same amount of the adenoviral-based biological delivery and expression system to the cells of the one or more intervertebral discs of (a), if monitoring of (c) shows that the degenerative disc disease in the intervertebral disc of the subject is not managed or treated; or

(e) further adjusting the amount of the adenoviral-based biological delivery and expression system and administering to the cells of one or more intervertebral discs of the subject in need thereof, of (a), if monitoring of (c) shows that the degenerative disc disease in the intervertebral disc of the subject has progressed.

46. The method of any one of embodiments 22-45, wherein the method further comprises administration of a corticosteroid and/or a local anesthetic into the intervertebral disc of the subject.

47. The method of embodiment 46, wherein the corticosteroid and/or local anesthetic is in a single pharmaceutical formulation with the adenoviral-based biological delivery and expression system such that the corticosteroid and/or local anesthetic are administered simultaneously with the adenoviral-based biological delivery and expression system.

48. The method of embodiment 46, wherein the corticosteroid and/or local anesthetic is not in a single pharmaceutical formulation with the adenoviral-based biological delivery and expression system such that the corticosteroid and/or local anesthetic are administered before and/or after the adenoviral-based biological delivery and expression system.

49. The method of any one of embodiments 22-48, wherein the method further comprises administration of a fluid into the intervertebral disc of the subject after the adenoviral-based biological delivery and expression system, optionally wherein the amount of fluid is, is about, is less than, is less than about, is more than, is more than about, 25, 30, 35, 40, 45, 50, 55, 60, 65, 70, 75, 80, 85, 90, 95, 100, 150, 200, 250, 300, 350, 400, 450, 500, 600, 700, 800, 900, 1000 μl, or a range defined by any two of the preceding values, optionally 25-1000, 25-500, 25-200, 50-150, 500-1000, 200-800, 200-600, 400-800, or 600-800 μl.

50. The method of embodiment 49, wherein the fluid is saline.

51. The method of any one of embodiments 46-50, wherein the further administration comprises intradiscal injection of the corticosteroid and/or local anesthetic and/or fluid.

52. The method of any one of embodiments 22-51, wherein the subject does not have Facet Joint Syndrome (FJS).

53. The method of any one of embodiments 22-51, wherein the subject has Facet Joint Syndrome (FJS).

54. The method of any one of embodiments 22-53, wherein the method comprises intradiscal injection of the pharmaceutical composition.

55. The method of embodiment 51 or 54, wherein the intradiscal injection is to the central gelatinous nucleus pulposus (NP) region.

56. The method of any one of embodiments 22-55, wherein the method does not comprises intra-tendinous, intra-muscular, intra-articular, or sub-acromial injection of the pharmaceutical composition.

57. The composition or method of any one of embodiments 1-56, wherein the concentration of the adenoviral-based biological delivery and expression system in the pharmaceutical formulation is, or is about, 1×10⁸ to 5×10¹¹ VP/ml, 2×10⁸ to 2×10¹¹ VP/ml, 2×10⁹ to 2×10¹¹ VP/ml, 1×10⁸ to 2×10⁹ VP/ml, or less than 1×10⁹ VP/ml.

58. The composition or method of any one of embodiments 1-56, wherein the concentration of the adenoviral-based biological delivery and expression system in the pharmaceutical formulation is, or is about, 1×10⁸ to 5×10¹¹ GC/ml, 2×10⁸ to 2×10¹¹ GC/ml, 2×10⁹ to 2×10¹¹ GC/ml, 1×10⁸ to 2×10⁹ GC/ml, or less than 1×10⁹ GC/ml.

59. The composition or method of any one of embodiments 1-58, wherein a single dose of the pharmaceutical composition administered to an intravertebral disc is an amount that is, is about, or is less than, 0.1, 0.2, 0.3, 0.4, 0.5, 1, 2, 3, 4, or 5 ml, or a range defined by any two of the preceding values, optionally 0.1 ml to 5 ml, 0.5 ml to 5 ml, 0.5 ml to 2 ml, 1 ml to 5 ml, 2 ml to 5 ml, 4 ml to 5 ml, or 3 ml to 5 ml.

60. The composition or method of any one of embodiments 17-59, wherein the infected cells comprise NP cells.

61. The composition or method of any one of the preceding embodiments, wherein the adenoviral-based biological delivery and expression system comprises, consists of, or consists essentially of, FX201/PCRX-201.

Unless otherwise defined, all technical and scientific terms used herein have the same meaning as commonly understood by one of ordinary skill in the art to which this disclosure belongs when read in light of the disclosure. In the disclosure, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The term “degenerative disc disease” as described herein is used interchangeably with the terms “discogenic low back pain”, “internal disc disruption” or intervertebral disc degeneration” or “degenerated intervertebral disc disease”.

Although methods and materials similar or equivalent to those described herein can be used in the practice or testing of the present disclosure, suitable methods and materials are described below. All publications, patent applications, patents, and other references mentioned herein are incorporated by reference in their entirety, and for the particular information discussed herein. The references cited herein are not admitted to be prior art to the claimed disclosure. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIG. 1 depicts the genome map of Humantakinogene Hadenovec (FX201, aka PCRX-201). ITR=inverted terminal repeats (1-103 bp on 5′; 29,158-29,260 bp on 3′), Ψ=packaging signals (240-375 bp), HPRT Stuffer=human hypoxanthine phosphoribosyltransferase (463-16,518 bp), Human Cosmid Insert=human cosmid (16,532-27,637 bp), SV40 Poly A=Simian virus 40 Poly A (27,750-28,020 bp), huIL-1Ra=human interleukin-1 receptor antagonist, the genome of interest (28,033-28,566 bp), NF-kB5-ELAM Promoter=NΦ-κB ινδυχιβλε promoter (28,581-28,842 bp).

FIG. 2 depicts the basic gene map of embodiments of the helper-dependent adenoviral vector of the disclosure. The vector backbone consists of the left and right inverted terminal repeats (ITR), adenoviral packaging signal (Ψ) and non-coding, non-viral stuffer sequences (remaining unmarked sequence between ITRs). The cDNA of human IL-1Ra, equine IL-1Ra (GQ-201), or murine IL-1Ra is cloned between the viral left and right ITRs of the used adenoviral-based vector. The expression of gene of IL-1-Ra is controlled by inflammation-sensitive NF-KB5-ELAM promoter.

FIGS. 3A-3D depict an embodiment of the metabolic activity of Human NP cells infected with PCRX-201 at a range of MOIs at 24 hr (FIG. 3A), 48 hr (FIG. 3B), 72 hr (FIG. 3C), and 1 week (FIG. 3D) post infection (*=P<0.05).

FIG. 4 depicts and embodiment of total DNA content per alginate bead culture measured using Pico green DNA quantification kit, following 21 days in alginate culture (*=P<0.05).

FIGS. 5A-5B depict embodiments of IL-1Ra production by monolayer human NP cells infected with PCRX-201 (aka FX201). IL-1Ra production in cell culture supernatant of monolayer-cultured human NP cells (from n=7 patients) is shown after 2 days, ±1 day stimulation with 10 ng IL-1β (FIG. 5A), and IL-1Ra production in cell culture supernatant after 5 days±4 days 10 ng IL-1β stimulation (FIG. 5B). The x-axis depicts the different stimulations, MOI of PCRX-201 and PCRX-201 plus Il-1β, as indicated, and the y-axis depicts the concentration of IL-1Ra in pg/ml in the cell culture supernatant, as determined by ELISA. The concentration of IL-1Ra produced by NP cells from each of the seven patients tested are represented dots, as indicated. The average level of IL-1Ra produced by NP cells, in a specific stimulation group is also indicated. Significance of difference of protein concentration between different stimulation groups is shown (*).

FIGS. 6A-6D depict embodiments of IL-1Ra production by 3D human NP cells infected with PCRX-201. Production of IL-1Ra by 3D-cultured human NP cells (from n=6 patients) infected with PCRX-201 (MOI of 0, 750 and 3000) in cell culture supernatant is shown after 2 days post infection (FIG. 6A), 7 days post infection (FIG. 6B), 14 days post infection (FIG. 6C), and 21 days post infection±7 days post stimulation with 10 ng IL-1β stimulation (FIG. 6D). The x-axis depicts the different stimulations, MOI of PCRX-201 and PCRX-201 plus IL-1β, as indicated, and the y-axis depicts the concentration of proteins in pg/ml in the cell culture supernatant, as determined by ELISA. The concentration of IL-1Ra produced by NP cells from each of the six patients tested are represented dots, as indicated. The average concentration of IL-1Ra produced by NP cells, in a specific stimulation group is also indicated. Significance of difference of protein concentration between different stimulation groups is shown (*).

FIGS. 7A-7H depicts an embodiment of long-term maintenance of IL-1Ra production from degenerate NP cells infected with PCRX-201. IL-1Ra production by NP cells isolated from degenerate disc tissue, from non-infected control cells and cells infected with PCRX-201 at MOI 3000. NP cells infected for 48 hrs prior to culture in 3D alginate beads for 2 days (FIG. 7A), 1 week (FIG. 7B), 2 weeks (FIG. 7C), 3 weeks (FIG. 7D), 4 weeks (FIG. 7E), 6 weeks (FIG. 7F), 8 weeks (FIG. 7G), 10 weeks (FIG. 7H), (*=P<0.05).

FIG. 8 depicts an embodiment of Relative gene expression for NGF normalized to 18s and untreated control in NP cells isolated from degenerate discs following 48 hr infection with PCRX-201 at MOI 750 or 3000 and cultured in alginate for 14 days prior to 100 pg/ml IL-13 stimulation for 1 further week.

FIGS. 9A-9D depict embodiments of VEGF production by 3D human NP cells infected with PCRX-201. Production of VEGF by 3D-cultured human NP cells (from n=6 patients) infected with PCRX-201 (MOI of 0, 750 and 3000) in cell culture supernatant is shown after 2 days post infection (FIG. 9A), 7 days post infection (FIG. 9B), 14 days post infection (FIG. 9C), and 21 days post infection±7 days post stimulation with 10 ng IL-1β stimulation (FIG. 9D). The x-axis depicts the different stimulations, MOI of PCRX-201 and PCRX-201 plus IL-1β, as indicated, and the y-axis depicts the concentration of proteins in pg/ml in the cell culture supernatant, as determined by ELISA. The concentration of VEGF produced by NP cells from each of the six patients tested are represented dots, as indicated. The average level of VEGF produced by NP cells, in a specific stimulation group is also indicated. Significance of difference of protein concentration between different stimulation groups is shown (*).

FIGS. 10A-10D depict embodiments of IL-1β production by 3D human NP cells infected with PCRX-201. Production of IL-1β by 3D-cultured human NP cells (from n=6 patients) infected with PCRX-201 (MOI of 0, 750 and 3000) in cell culture supernatant is shown after 2 days post infection (FIG. 10A), 7 days post infection (FIG. 10B), 14 days post infection (FIG. 10C), and 21 days post infection±7 days post stimulation with 10 ng IL-1β stimulation (FIG. 10D). The x-axis depicts the different stimulations, MOI of PCRX-201 and PCRX-201 plus IL-1β, as indicated, and the y-axis depicts the concentration of proteins in pg/ml in the cell culture supernatant, as determined by ELISA. The concentration of IL-13 produced by NP cells from each of the six patients tested are represented dots, as indicated. The average level of IL-1β produced by NP cells, in a specific stimulation group is also indicated. Significance of difference of protein concentration between different stimulation groups is shown (*).

FIGS. 11A-11C depict embodiments of IL-6 production by 3D human NP cells infected with PCRX-201. Production of IL-6 by 3D-cultured human NP cells (from n=6 patients) infected with PCRX-201 (MOI of 0, 750 and 3000) in cell culture supernatant is shown after 7 days post infection (FIG. 11A), 14 days post infection (FIG. 11B), and 21 days post infection±7 days post stimulation with 10 ng IL-1β stimulation (FIG. 11C). The x-axis depicts the different stimulations, MOI of PCRX-201 and PCRX-201 plus IL-1β, as indicated, and the y-axis depicts the concentration of proteins in pg/ml in the cell culture supernatant, as determined by ELISA. The concentration of IL-6 produced by NP cells from each of the six patients tested are represented dots, as indicated. The average level of IL-6 produced by NP cells, in a specific stimulation group is also indicated. Significance of difference of protein concentration between different stimulation groups is shown (*).

FIGS. 11D-11F depict embodiments of IL-8 production by 3D human NP cells infected with PCRX-201. Production of IL-8 by 3D-cultured human NP cells (from n=6 patients) infected with PCRX-201 (MOI of 0, 750 and 3000) in cell culture supernatant is shown after 7 days post infection (FIG. 11D), 14 days post infection (FIG. 11E), and 21 days post infection±7 days post stimulation with 10 ng IL-1β stimulation (FIG. 11F). The x-axis depicts the different stimulations, MOI of PCRX-201 and PCRX-201 plus IL-1β, as indicated, and the y-axis depicts the concentration of IL-8 in pg/ml in the cell culture supernatant, as determined by ELISA. The concentration of IL-8 produced by NP cells from each of the six patients tested are represented dots, as indicated. The average level of IL-8 produced by NP cells, in a specific stimulation group is also indicated. Significance of difference of protein concentration between different stimulation groups is shown (*).

FIG. 12A depicts an embodiment of MMP3 production by 3D human NP cells infected with PCRX-201. Production of MMP3 by 3D-cultured human NP cells (from n=6 patients) infected with PCRX-201 (MOI of 0, 750 and 3000) in cell culture supernatant is shown after 21 days post infection±7 days post stimulation with 10 ng IL-1β stimulation (FIG. 12A). The x-axis depicts the different stimulations, MOI of PCRX-201 and PCRX-201 plus IL-1β, as indicated, and the y-axis depicts the concentration of MMP3 in pg/ml in the cell culture supernatant, as determined by ELISA. The concentration of MMP3 produced by NP cells from each of the six patients tested are represented dots, as indicated. The average level of MMP3 produced by NP cells, in a specific stimulation group is also indicated. Significance of difference of protein concentration between different stimulation groups is shown (*).

FIG. 12B depicts an embodiment of ADAMTS4 production by 3D human NP cells infected with PCRX-201. Production of ADAMTS4 by 3D-cultured human NP cells (from n=6 patients) infected with PCRX-201 (MOI of 0, 750 and 3000) in cell culture supernatant is shown after 21 days post infection±7 days post stimulation with 10 ng IL-1β stimulation (FIG. 12B). The x-axis depicts the different stimulations, MOI of PCRX-201 and PCRX-201 plus IL-1β, as indicated, and the y-axis depicts the concentration of proteins in pg/ml in the cell culture supernatant, as determined by ELISA. The concentration of ADAMTS4 produced by NP cells from each of the six patients tested are represented dots, as indicated. The average level of ADAMTS4 produced by NP cells, in a specific stimulation group is also indicated. Significance of difference of protein concentration between different stimulation groups is shown (*).

FIGS. 13A-13E depicts embodiments of paracrine effects of PCRX-201 infected cells. Protein production for IL-1Ra (FIG. 13A), IL-1β (FIG. 13B), IL-6 (FIG. 13C), MMP 3 (FIG. 13D), ADAMTS4 (FIG. 13E) following treatment of human NP cells derived from degenerate discs treated+/−conditioned media from patient matched conditioned media from PCRX-201 infected cells (*=P<0.05).

FIG. 14 depicts an embodiment of a human NP tissue explant in a semi-constrained culture system.

FIGS. 15A-15B depict embodiments of IL-1Ra protein concentration released into media (FIG. 15A), and number of cells with immunopositive staining for IL-1Ra in human NP explants from degenerate IVD samples (FIG. 15B) injected with PCRX-201 at ˜MOI 3000 together with non-injected controls (*=P<0.05).

FIGS. 16A-16G depict embodiments of the concentration of VEGF (FIG. 16A), IL-1β (FIG. 16B), IL-6 (FIG. 16C), MMP 3 (FIG. 16D), ADAMTs4 (FIG. 16E), Collagen type II (FIG. 16F) and aggrecan (FIG. 16G) released into media from human NP explants from degenerate IVD samples injected with PCRX-201 at ˜MOI 3000 together with non-injected controls (*=P<0.05).

FIGS. 17A-17F depict embodiments of percentage immunopositivity for VEGF (FIG. 17A), NGF (FIG. 17B), IL-1β (FIG. 17C), MMP 3 (FIG. 17D), ADAMTs4 (FIG. 17E) and collagen type II (FIG. 17F) in human NP explants from degenerate IVD samples injected with PCRX-201 at ˜MOI 3000 together with non-injected controls (*=P<0.05).

### DETAILED DESCRIPTION OF THE INVENTION

The present disclosure provides compositions for an improved delivery and expression system that allows for a long-term expression of biologically active recombinant interleukin-1 receptor antagonist (IL-1Ra) in cells of intervertebral disc, including those of subjects suffering from degenerative disc disease (DDD) or a condition associated with DDD. In some embodiments, these compositions are used for the treatment of degenerative disc disease (DDD) or a condition associated with DDD. Disclosed herein is a novel IL-1Ra gene therapy (FX201, humantakinogene hadenovec, also referred to as PCRX-201), for administration to intervertebral disc, that is being developed for the treatment of patients of DDD or a condition associated with DDD. FX201 (humantakinogene hadenovec, PCRX-201), is a helper-dependent adenovirus (HDAd) delivering a nucleic acid sequence encoding the human IL-1Ra under the control of a nuclear factor-κB (NF-κB)-inducible promoter for administration to intervertebral disc of patients with DDD or a condition associated with DDD. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

The terms “treatment of degenerative disc disease (DDD) or a condition associated with DDD” or “Treatment of DDD” as described herein has its ordinary and customary meaning as read in light of this disclosure, and does not encompass prevention of onset or development of DDD in the one or more intervertebral discs of the subject suffering therefrom, but encompasses inhibition or abrogation of progress of an existing DDD or condition associated with DDD in the one or more intervertebral discs of the subject suffering therefrom. In some aspects, the “treatment of degenerative disc disease (DDD) or a condition associated with DDD” or “Treatment of DDD” also encompass reversal of or recovery from DDD or a condition associated with DDD in the one or more intervertebral discs of the subject suffering therefrom. The term “prevention” has its ordinary and customary meaning as read in light of this disclosure, and as known in the art encompasses preventing or stopping the development of DDD or characteristics/conditions known to be associated with DDD, in the one or more intervertebral discs of a subject in need thereof, wherein the one or more intervertebral discs of the subject do not yet exhibit DDD or characteristics/conditions known to be associated with DDD.

The present disclosure provides compositions for intervertebral disc injection (i.e., intradiscal injection) of an adenoviral-based biological delivery and expression system (an adenoviral-based vector) comprising a nucleic acid sequence encoding a mammalian (e.g. human) interleukin-1 receptor antagonist (IL-1Ra) protein, for example FX201. Following intervertebral disc injection, the adenoviral delivery and expression system infects cells in the intervertebral disc to produce IL-1Ra locally in response to inflammation. In some embodiments, the adenoviral delivery and expression system (e.g., FX201) is a non-replicating, non-integrating HDAd vector with no viral coding sequences that has been engineered to carry the genetic coding sequence for IL-1Ra (e.g., human IL-1Ra). In some embodiments, only the adenoviral packaging signal and inverted terminal repeats (ITRs) remain in the adenovirus genome as they are required for manufacturing. In some embodiments, transcription is controlled by the inflammation-sensitive NF-κB-inducible promoter, which drives expression of IL-1Ra in response to an inflammatory environment. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

In some embodiments, the adenoviral delivery and expression system is administered as a single dose by intervertebral disc injection (intradiscal). The expected clinical benefits are sustained symptomatic relief, including both reduction in pain and improvement or restoration of function, and a beneficial modification of the underlying disease process in patients with DDD or a condition associated with DDD. Advantageously, in some embodiments the adenoviral delivery and expression systems of the present disclosure specifically locates in the cells of intervertebral disc when administered to the intervertebral disc. Therefore, in some embodiments IL-1Ra concentrations are highest in the intervertebral disc injected with the vector of the disclosure while no significant side effects are present in any other organ.

In some embodiments, the adenoviral delivery and expression system of the compositions disclosed herein, is the same as disclosed in the PCT Application No: PCT/US2020/051642 (Publication No: WO2021/055860), the entire contents of which are incorporated herein by reference. In some embodiments, the adenoviral delivery and expression system of the compositions disclosed herein, is the same as disclosed in the PCT Application No: PCT/IB2013/000198 (Publication No: WO2013/114199), the entire contents of which are incorporated herein by reference. Described below are the properties of embodiments of the adenoviral delivery and expression system.

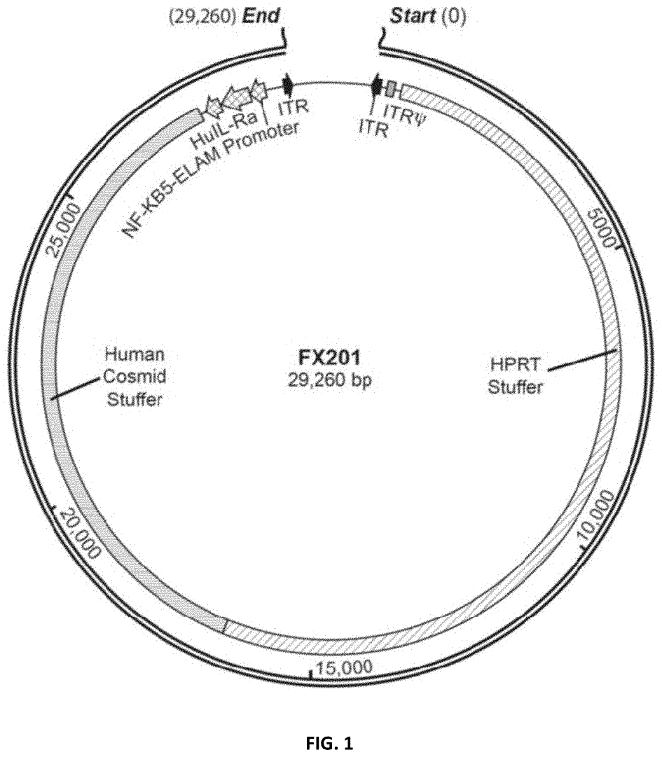

Vector Backbone: In some embodiments, (e.g., FX201) the adenoviral delivery and expression system is a non-replicating, non-integrating HDAd vector. The genomic component is composed of double-stranded linear DNA approximately 29.3 kilobases (kb) in size. In some embodiments, (e.g., FX201) the adenoviral delivery and expression system genome contains minimal adenoviral elements required for amplification and packaging to allow for its manufacturing: left and right inverted terminal repeats (hereafter referred to as “L ITR” and “R ITR”, respectively) and the packaging signal (Ψ). In some embodiments, approximately 1.1 kb of the adenoviral delivery and expression system (e.g., FX201) genome is composed of a nucleic acid sequence encoding human IL-1Ra, which is inserted on the right end of the genome in reverse (right-to-left) orientation, and the promoter, placed just before the R ITR. In some embodiments, the promoter is 5 species-conserved NF-κB binding motif repeats fused to a proximal promoter region of the human ELAM gene, responding to pro-inflammatory cytokines (Schindler 1994). In some embodiments, approximately 27 kb of the adenoviral delivery and expression system (e.g., FX201) genome consists of non-coding stuffer sequence composed of human hypoxanthine phosphoribosyltransferase (HPRT) and human cosmid insert, which is inserted to enlarge the genome to a size which allows efficient packaging of the vector genome into each viral particle. A genome map for an embodiment of the adenoviral delivery and expression system, FX201, is presented in FIG. 1.

Gene of Interest: In some embodiments, (e.g., FX201) the adenoviral delivery and expression system genome contains a 534 base pair (bp) sequence of human IL-1Ra, which is regulated by a 262 bp sequence of NF-κB-inducible promoter.

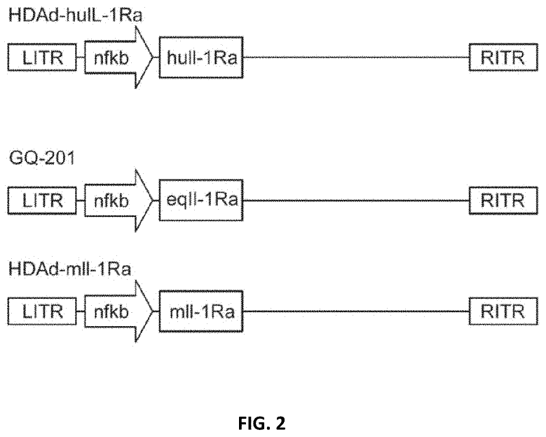

Disclosed herein are gene maps of the FX201, and embodiments of HDAd vectors of the disclosure (FIG. 2). The HdAd vector of the present disclosure can contain the inflammation-sensitive NF-κB5-ELAM promoter upstream of the IL-1Ra cDNA according to any one of SEQ ID NOs: 1 or 4, as well as ITR and an adenoviral packaging signal. The full vector sequence of HDAd-mIL-1Ra, GQ-201, and HDAd-human IL-1Ra, is shown in SEQ ID NOs: 2, 3 and 7 respectively. The only difference between the three vectors is that GQ-201 carries the equine variant of IL-1Ra, HDAd-mIL-Ra has the murine IL-1Ra variant and HDAd-huIL-1Ra carries the human IL-1Ra. As an example, the HDAd-mIL-Ra of nucleic acid sequence according to SEQ ID NO: 3 can comprise a nucleic acid encoding a murine IL-1Ra according to SEQ ID NO: 1. As an example, the HDAd-human IL-Ra of nucleic acid sequence according to SEQ ID NO: 7 can comprise a nucleic acid encoding a human IL-1Ra according to SEQ ID NO: 4. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

In some embodiments, the vectors disclosed herein are cloned by standard digestion/ligation reactions according to the following strategy. The luciferase cDNA in pNifty-luc, a plasmid that contains the luciferase cDNA driven by a NF-κB5-ELAM promoter, was excised with Ncol and Nhel and cDNAs for equine, murine or human IL-1Ra were ligated into this position. The NF-κB5-ELAM promoter—murine IL-1Ra or NF-κB5-ELAM promoter—equine IL-1Ra or NF-κB5-ELAM promoter—human IL-1Ra cassettes were excised with Notl and Pad or EcoRI and Pad, blunted and inserted into pLPBL shuttle plasmid, which had been linearized with Sail and blunted. The NF-κB5-ELAM promoter—murine IL-1Ra or NF-κB5-ELAM promoter—equine IL-1Ra or NF-κB5-ELAM promoter—human IL-1Ra cassettes were then excised with Ascl, which flanks both sides of the multiple cloning site, and ligated into Ascl linearized ρΔ28 plasmid (Toietta, G., Pastore, L, Cerullo, V., Finegold, M., Beaudet, A. L., and Lee, B. (2002). Generation of helper-dependent adenoviral-based vectors by homologous recombination. Mol Ther 5, 204-210.), which yielded the genomic plasmids pA28-mll-1Ra, pA28-eqll-1Ra and pA28-hull-1Ra. These plasmids were digested with Pmel in order to linearize the vector, liberate the inverted terminal repeats and excise bacterial resistance genes. Vectors were rescued and amplified as described before using the helper-virus AdNG163R-2 and 116 cell factories (Palmer, D., and Ng, P. (2003). Improved system for helper-dependent adenoviral vector production. Mol Ther 8, 846-852; Suzuki, M., Cela, R., Clarke, C, Bertin, T. K., Mourino, S., and Lee, B. (2010). Large-scale production of high-quality helper-dependent adenoviral-based vectors using adherent cells in cell factories. Hum Gene Ther 21, 120-126.)

### Compositions of the Present Disclosure

In some embodiments, compositions of the present disclosure can comprise adenoviral-based biological delivery and expression systems based on a adenoviral-based vectors, wherein the adenoviral-based vectors comprise a nucleic acid sequence encoding for human or mammalian interleukin-1 receptor antagonist (IL-1Ra), L ITR, R ITR, adenoviral packaging signal and non-viral, non-coding stuffer nucleic acid sequences. In some embodiments, the nucleic acid sequence encoding for IL-1Ra contains the cDNA sequence of Il-1Ra selected from the group consisting of murine Il-1Ra, equine 1-1Ra, canine Il-1Ra, cat Il-1Ra, rabbit Il-1Ra, hamster Il-1Ra, bovine Il-1Ra, camel Il-1Ra and their homologs in other mammalian species. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

In some embodiments, the adenoviral-based vectors of the present disclosure can minimize immune responses in the host and confer long-term gene expression of human or mammalian IL-1Ra in intervertebral discs, including those that are affected by DDD. In some embodiments, the adenoviral-based vector of the present disclosure is a helper-dependent adenoviral-based vector (HDAd).

In some embodiments, the sequence encoding for the human or mammalian interleukin-1 receptor antagonist (IL-1Ra) in the compositions of the present disclosure is controlled by an inflammation-sensitive promoter. Without wishing to be bound by theory, the use of an inflammation-sensitive promoter in the compositions of the present disclosure provides for specific control of IL-1Ra gene expression in tissue and cells of intervertebral disc suffering from DDD or conditions associated with DDD, as only cells that are affected by the disease or conditions associated with the disease, will express and secrete the IL-1Ra gene product, whereas cells that are not affected will not express and secret the IL-1Ra. In some aspects, the promoter sequences is located upstream of the reading frame of the sequence encoding for the human or mammalian IL-1Ra.

In some embodiments, the inflammation-sensitive promoters used in the compositions of the present disclosure is specifically activated by increased levels of factors including immune stimulatory substances and/or cytokines. Without wishing to be bound by theory, during DDD or conditions associated with DDD, a variety of immune stimulatory substances and cytokines are released, resulting in high levels of promoter-activating substances. In a non-limiting example, an immune stimulatory substance is IL-1, which is known to play a pivotal role in development and pathogenesis of DDD, and in inducing intervertebral disc matrix degradation. The released immune stimulatory substances and/or cytokines can activate transcription factors such as NF-κB, which regulates the NF-κB promoter. Therefore, the release of such DDD or DDD associated condition-specific immune stimulatory substances and/or cytokines can allow for the control of gene expression in intervertebral discs of humans or mammals suffering from DDD and conditions associated with DDD. In some embodiments, the release of such DDD or DDD associated condition-specific immune stimulatory substances and/or cytokines can allow for the control of gene expression in intervertebral discs of humans or mammals for treating or preventing DDD and conditions associated with DDD.

In some embodiments, only the cells of the degenerate discs infected with the adenoviral-based biological delivery and expression system produce IL-1Ra. In some embodiments, the IL-1Ra produced by the cells of the degenerate discs infected with the adenoviral-based biological delivery and expression system, acts on IL-1 receptor of intervertebral disc cells in an autocrine manner. In some embodiments, the IL-1Ra produced by the cells of the degenerate discs infected with the adenoviral-based biological delivery and expression system, acts on IL-1 receptor of intervertebral disc cells and/or cells of tissues surrounding the intervertebral disc, in an paracrine manner. The term “autocrine” as described herein has its ordinary and customary meaning as read in light of this disclosure, and is when a molecule produced by a cell acts on the cell producing the factor itself. The term “paracrine” as described herein has its ordinary and customary meaning as read in light of this disclosure, and is when a molecule produced by a cell acts on the cells other than itself, in the surrounding tissue. In some embodiments, the IL-1Ra produced by the cells of the degenerate discs infected with the adenoviral-based biological delivery and expression system, acts on IL-1 receptor of cells of the same degenerate intervertebral discs. In some embodiments, the IL-1Ra produced by the cells of the degenerate discs infected with the adenoviral-based biological delivery and expression system, acts on IL-1 receptor of cells of the other degenerated intervertebral discs in the surrounding. In some embodiments, the IL-1Ra produced by the cells of the degenerate discs infected with the adenoviral-based biological delivery and expression system, acts on IL-1 receptor of cells of the other non-degenerate intervertebral discs in the surrounding. In some embodiments, the IL-1Ra produced by the cells of the degenerate discs infected with the adenoviral-based biological delivery and expression system, acts directly or indirectly on cells of tissues surrounding the degenerate discs infected with the adenoviral-based biological delivery and expression. In some embodiments the tissues surrounding the degenerate discs is a blood vessel, a nervous tissue, a muscle, a ligament, a tendon, a skeletal tissue (e.g. a vertebra) or a spinal cord tissue or a combination thereof. The blood vessel can be a blood vessel carrying blood to or from a vertebral column or any branches thereof. The nervous tissue can be a nerve innervating a vertebral column or any branches thereof.

It is contemplated that any inflammation-sensitive promoter can be used in context of the present disclosure. In some embodiments, it is contemplated that the inflammation-sensitive promoter results in a specific expression of the IL-1Ra gene product in cells and tissue of intervertebral disc with DDD or condition associated with DDD. In some embodiments, the inflammation-sensitive promoter for use in the present disclosure includes, but is not limited to promoters inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), macrophage inflammatory protein-1a (MIP-1a), or hybrid constructs of the above, or otherwise disclosed herein.

In some embodiments, the inflammation-sensitive promoter is an NF-κB5-ELAM promoter. The NF-κB-inducible promoter, composed of five species-conserved NF-κB binding motif repeats fused to a proximal promoter region of the human endothelial leukocyte adhesion molecule (ELAM) gene, was chosen to drive the expression of IL-1Ra for several reasons. First, NF-κB, as a transcription factor, is ubiquitously expressed in all cells of the body, and any transduced cell, when stimulated with inflammatory cues, can in principle express the IL-1Ra transgene. Therefore, there is no cell specificity requirement to induce IL-1Ra expression. Additionally, NF-κB is the terminal signaling molecule for receptors of pro-inflammatory cytokines, such as interleukin-1 (IL-1) and tumor necrosis factor-α and other immune cell receptors such as toll-like receptors, where it acts to initiate a cellular response to many pro-inflammatory inputs. As such, the activation of IL-1Ra production is designed to be stimulated in the intervertebral disc by a variety of inflammatory signals. In some embodiments, the inflammation-sensitive promoter shares one or more of these features with the NF-κB-inducible promoter.

In some embodiments, following intervertebral disc injection, the gene of IL-1Ra is delivered to intervertebral disc cells, including, but not limited to the central gelatinous nucleus pulposus (NP) region (nucleus pulposus (NP) cells). In some embodiments, following injection, NP cells that are affected by inflammation start to produce recombinant IL-1Ra protein under the control of the inflammation-sensitive promoter (e.g. the NF-κB promoter). In some embodiments, high amounts of IL-1Ra are then secreted into the intervertebral disc space, where IL-1Ra inhibits inflammation and catabolic proteins, and/or stops or reduces cartilage degradation by blocking the interleukin-1 receptor on the surface of NP cells and other cells embedded in the intervertebral disc region and space. In some embodiments, high local concentrations of recombinant IL-1Ra does not result in any adverse side effects.

In some embodiments, as disclosed herein, intervertebral disc degradation and proteins involved in pathogenesis of intervertebral disc of DDD are inhibited effectively using the adenoviral-based biological delivery and expression system of the present disclosure. In some embodiments, high local and low systemic concentrations of the therapeutic protein IL-1Ra is achieved through administration of the compositions of the present disclosure. In some embodiments, the result is high efficacy in the treatment of DDD with no or minimal side effects.

In some embodiments, the cells of one or more intervertebral discs of the subject in need thereof, infected with the adenoviral-based biological delivery and expression system express IL-1Ra for a period of at least 2 weeks, at least 1 month, at least 3 months, at least 6 months or at least 1 year. Consequently, in some embodiments, medical and economic burden associated with frequent intervertebral disc injections required for short-term treatments are significantly reduced. In some embodiments, potential complications associated with treatment for degenerated disc disorder are minimized and intervertebral disc health is preserved resulting in sustained health improvement of the treated subject (e.g., human).

In some embodiments, the inflammation-sensitive IL-1Ra production of the adenoviral-based biological delivery and expression system (adenoviral-based vectors) of the disclosure allows for the prevention of the development of an DDD as cells in the intervertebral disc that are infected with the adenoviral-based vector of the disclosure remain do not express the IL-1Ra gene in the absence of immune stimulatory substances that could activate the NF-κB5-ELAM promoter or any other inflammation-sensitive promoter. Only if the pathogenesis of DDD initiates, the promoter is activated as a result of inflammation and subsequently IL-1Ra is produced and secreted. Thus, by using the adenoviral delivery and expression system of the disclosure, this mechanism allows for the prevention of the development of DDD in an early stage.

In some embodiments, the inflammation-sensitive IL-1Ra production of the adenoviral-based vectors of the disclosure results in IL-1Ra no longer be produced when the DDD condition is resolved or has disappeared. As a result, in some embodiments, the inflammation-sensitive IL-1Ra production of the adenoviral-based vectors of the disclosure are safer for administration to a subject.

In some embodiments, the adenoviral-based vectors of the present disclosure does not carry any viral sequences, except for the L ITR, R ITR and the adenoviral packaging signal. In some embodiments, adenoviral-based vectors used in the present disclosure are helper-dependent adenoviral-based vectors based on the helper virus and helper-dependent backbone system developed by Palmer and Ng (Palmer, D., and Ng, P. (2003). Improved system for helper-dependent adenoviral vector production. Mol Ther 8, 846-852.) and Toietta et al (Toietta, G., Pastore, L., Cerullo, V., Finegold, M., Beaudet, A. L., and Lee, B. (2002). Generation of helper-dependent adenoviral-based vectors by homologous recombination. Mol Ther 5, 204-210.). In some embodiments, an adenoviral delivery and expression system according to the present disclosure can comprise a nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences. In some embodiments, adenoviral delivery and expression system comprises or consists of the nucleic acid sequence as set forth in SEQ ID NO: 2, SEQ ID NO: 3 or SEQ ID NO: 7, or a biologically effective part thereof. The nucleic acid sequence of SEQ ID NO 2 describes a murine helper-dependent adenoviral-based vector, the sequence set forth in SEQ ID NO 3 describes an equine helper-dependent adenoviral-based vector, the sequence set forth in SEQ ID NO 7 describes a human helper-dependent adenoviral-based vector, all three vectors bearing any one of a murine IL-1Ra gene, an equine IL-1Ra gene or human IL-1Ra gene respectively. In some embodiments, the system of the disclosure has any one of at least 96%, 97%, 98%, or 99% sequence homology with the vector set forth in SEQ ID NO: 2, SEQ ID NO: 3 or SEQ ID NO: 7. In some embodiments, the adenoviral delivery and expression system comprises or consists of a nucleic acid sequence that is, or is at least, 80%, 85%, 90%, 95%, 96%, 97%, 98%, 99% or 100% identical to the vector set forth in SEQ ID NO: 2, SEQ ID NO: 3 or SEQ ID NO: 7. In some embodiments of the adenoviral delivery and expression system, the nucleic acid sequence encoding IL-1Ra comprises or consists of a nucleic acid sequence that is, or is at least, 80%, 85%, 90%, 95%, 96%, 97%, 98%, 99% or 100% identical to the nucleic acid of SEQ ID NO 4. In some embodiments of the adenoviral delivery and expression system, the nucleic acid sequence encoding IL-1Ra comprises or consists of a nucleic acid sequence that encodes an IL-1Ra protein comprising or consisting of an amino acid sequence that is, or is at least, 80%, 85%, 90%, 95%, 96%, 97%, 98%, 99% or 100% identical to the amino acid sequence of SEQ ID NO: 6.

In some embodiments, the present disclosure provides a pharmaceutical composition, and methods of using the same, for infection of an intervertebral disc(s) cell(s), e.g. an NP cell, of a subject identified as having or at risk of developing, DDD or a condition associated with DDD, which comprises an adenoviral-based biological delivery and expression system comprising a nucleic acid sequence encoding a human interleukin-1 receptor antagonist (IL-1Ra) protein. In some embodiments, the a nucleic acid sequence further comprises left and right inverted terminal repeats, an adenoviral packaging signal and non-viral, and non-coding stuffer nucleic acid sequences. In some embodiments, the expression of the human IL-1Ra gene is regulated by a NF-kB inducible promoter, which is located upstream of the reading frame of the nucleic acid sequence encoding the human IL-1Ra protein. In some embodiments, the human IL-1Ra gene is regulated by an inflammation-sensitive promoter other than the NF-kB inducible promoter. In some embodiments, the inflammation-sensitive promoter is selected from the group consisting of a promoter inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), macrophage inflammatory protein-1a (MIP-1a), or hybrid constructs of the above, or otherwise disclosed herein. In some embodiments, the adenoviral delivery and expression system of the pharmaceutical composition for treatment of DDD or a condition associated with DDD, in a subject in need thereof, comprises or consists of a nucleic acid sequence that is, or is at least, 80%, 85%, 90%, 95%, 96%, 97%, 98%, 99% or 100% identical to the vector set forth in SEQ ID NO: 2, SEQ ID NO: 3 or SEQ ID NO: 7. In some embodiments, the pharmaceutical formulation comprises an effective amount of the adenoviral-based biological delivery and expression system to neutralize the effect of IL-1 on intervertebral disc inflammation and catabolic activity in the subject, and/or to provide improvement in one or more measures of DDD or a condition associated with DDD in the subject. In some embodiments, the infection of an intervertebral disc(s) cell(s), e.g. an NP cell, is performed by intradiscal injection into the intervertebral disc of the pharmaceutical composition. In some embodiments, the injection is not intra-articular.

In some embodiments, the present disclosure provides a pharmaceutical composition for treatment of DDD or a condition associated with DDD, in a subject in need thereof, which comprises an effective amount of an adenoviral-based biological delivery and expression system comprising a nucleic acid sequence encoding a human interleukin-1 receptor antagonist (IL-1Ra) protein, left and right inverted terminal repeats, an adenoviral packaging signal and non-viral, and non-coding stuffer nucleic acid sequences, wherein the expression of the human IL-1Ra gene is regulated by a NF-kB inducible promoter, which is located upstream of the reading frame of the nucleic acid sequence encoding the human IL-1Ra protein, and wherein the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 95% homologous or identical to the nucleic acid sequence of SEQ ID NO: 7. In some embodiments, the human IL-1Ra gene is regulated by an inflammation-sensitive promoter other than the NF-kB inducible promoter. In some embodiments, the inflammation-sensitive promoter is selected from the group consisting of a promoter inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), macrophage inflammatory protein-1a (MIP-1a), or hybrid constructs of the above, or otherwise disclosed herein. In some embodiments, the adenoviral delivery and expression system of the pharmaceutical composition for treatment of DDD or a condition associated with DDD, in a subject in need thereof, comprises or consists of a nucleic acid sequence that is, or is at least, 80%, 85%, 90%, 95%, 96%, 97%, 98%, 99% or 100% identical to the vector set forth in SEQ ID NO: 2, SEQ ID NO: 3 or SEQ ID NO: 7. In some embodiments the subject has Facet Joint Syndrome (FJS). In some embodiments the subject does not have Facet Joint Syndrome (FJS).

In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 95%, at least 96%, at least 97%, at least 98% or at least 99% homologous or identical to the nucleic acid sequence of SEQ ID NO: 7.

In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 99% homologous to the nucleic acid sequence of SEQ ID NO: 7. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is identical to the nucleic acid sequence of SEQ ID NO: 7.

“Long term expression” has its ordinary and customary meaning as read in light of this disclosure, and in the context of the present disclosure includes wherein the gene product of the adenoviral delivery and expression system (i.e. IL-1Ra), is expressed in the intervertebral disc(s) infected with the adenoviral-based vector (adenoviral-based biological delivery and expression system) of the disclosure, for at least 3 months, at least 6 months or at least 12 months. In some embodiments, the IL-1Ra is expressed in the intervertebral disc(s) infected with the adenoviral-based vector of the disclosure for at least 3 months. In some embodiments, the IL-1Ra is expressed in an intervertebral disc(s) cell, e.g. an NP cell, infected with the adenoviral-based vector of the disclosure for a period that is, is about, is at least, is at least about, 1, 2, 3, 4, 5, 6, 7, 8, 9, 10, 11, 12, 16, 20, 24, or 52 weeks, or a range defined by any two of the preceding values, for example 1-24, 1-52, 4-12, 4-24, 8-12, 8-24, 12-24, or 8-16 weeks.

“Biologically effective” has its ordinary and customary meaning as read in light of this disclosure, and in the context of the present disclosure includes wherein the gene product of the adenoviral delivery and expression system comprises the full or partial polypeptide sequence of IL-1Ra having the in-intervertebral disc activity to neutralize the effect of IL-1 on intervertebral disc inflammation and catabolic activity, and/or to provide improvement in one or more measures of DDD or a condition associated with DDD.

In some embodiments, the adenoviral-based vector (adenoviral-based biological delivery and expression system) of the disclosure comprises a nucleic acid sequence of IL-1Ra under control of an inflammation-sensitive promoter. Although IL-1Ra contains species-specific nucleic acid sequences, in some embodiments the adenoviral-based vector is able to express interleukin-1 receptor antagonist (IL-1Ra) from any mammalian species or human. In some embodiments, the cDNA of the mammalian interleukin-1 receptor antagonist (IL-1Ra) used for cloning and included in the adenoviral-based vector is a cDNA selected from the group consisting of human IL-1Ra, murine IL-1Ra, equine IL-1Ra, canine IL-1Ra, cat II-1Ra, rabbit IL-1Ra, hamster IL-1Ra, bovine IL-1Ra, camel IL-1Ra or their homologs in other mammalian species. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

In some embodiments, in order to monitor the presence of genomic vector sequences in cells of the injected intervertebral disc, the adenoviral-based vector (adenoviral-based biological delivery and expression system) according to the disclosure can further comprise a sequence encoding a marker gene that is visually or instrumentally detectable. In some embodiments, the marker gene is selected from the group, but is not limited to, green fluorescence protein (GFP), LacZ, and luciferase enzyme.

In some embodiments, as an example, the nucleic acid sequence of murine IL-1Ra as used in the present disclosure is shown in the sequence listing set forth in SEQ ID NO: 1. As noted above, and as otherwise disclosed herein, any nucleic acid sequence resulting in a biologically active IL-1Ra protein of any mammalian or human species can be used in the context of the present disclosure. Furthermore, conserved nucleic acid sequences encoding for the same amino acids, polypeptide or protein fall under scope of the present disclosure. In some embodiments, the adenoviral-based vector according to the disclosure contains a nucleic acid sequence (e.g. cDNA) of IL-1Ra having at least 95%, 96%, 97%, 98% or 99% sequence homology with the nucleic acid sequence shown in SEQ ID NO: 1. In some embodiments, the disclosure also comprises biologically active nucleic acid sequences of IL-1Ra or fragments thereof. Thus, in some embodiments, the help-dependent adenoviral-based vectors of the present disclosure can comprise a biologically active fragment of the nucleic acid sequence put forth in SEQ ID NO: 1.

In some embodiments, as an example, the nucleic acid sequence of human IL-1Ra as used in the present disclosure is shown in the sequence listing set forth in SEQ ID NO: 4. As noted above, and otherwise herein, in some embodiments, a nucleic acid sequence resulting in a biologically active IL-1Ra protein of a human can be used in the context of the present disclosure. In some embodiments, conserved nucleic acid sequences encoding for the same amino acids, polypeptide or protein fall under scope of the present disclosure. In some embodiments, the adenoviral-based vector according to the disclosure contains a nucleic acid sequence (e.g. cDNA) of IL-1Ra having at least 95%, 96%, 97%, 98% or 99% sequence homology with the nucleic acid sequence shown in SEQ ID NO: 4. In some embodiments, the disclosure also comprises biologically active nucleic acid sequences of IL-1Ra or fragments thereof. Thus, in some embodiments, the adenoviral-based vectors of the present disclosure can comprise a biologically active fragment of the nucleic acid sequence put forth in SEQ ID NO: 4.

In some embodiments, as an example, the nucleic acid sequence of human IL-1Ra as used in the present disclosure can express a human IL-1Ra protein of amino acid sequence that is at least 95% homologous or identical to SEQ ID NO: 6. In some embodiments, the nucleic acid sequence of human IL-1Ra as used in the present disclosure can express a human IL-1Ra protein of amino acid sequence that is at least 96%, 97%, 98% or 99% homologous or identical to SEQ ID NO: 6. The nucleic acid sequence of human IL-1Ra as used in the present disclosure as set forth in SEQ ID NO: 4 can express a human IL-1Ra protein of amino acid sequence that is at least 99% homologous or identical to SEQ ID NO: 6. In some embodiments, the nucleic acid sequence of human IL-1Ra as used in the present disclosure can express a human IL-1Ra protein of amino acid sequence according to SEQ ID NO: 6.

In some embodiments, the human-IL-1Ra can have an amino acid sequence that is at least 95% to 99% homologous or identical to the amino acid sequence of a wild type human IL-1Ra protein. In some embodiments, the human-IL-1Ra can have an amino acid sequence that is 95% to 99% homologous or identical to a human IL-1Ra protein of amino acid sequence according to SEQ ID NO: 6.

In some embodiments, the present disclosure provides an adenoviral-based biological delivery and expression system for the expression of IL-1Ra in cells of an intervertebral disc, and/or for the treatment of DDD or a condition associated with DDD or for the prevention of such conditions, in a human identified to be suffering from or at risk of developing DDD or an DDD condition, wherein the adenoviral-based biological delivery and expression system comprises genome copies (GC) of a adenoviral-based vector comprising a nucleic acid sequence encoding a human interleukin-1 receptor antagonist (IL-1Ra). In some embodiments, the nucleic acid further comprises left and right inverted terminal repeats, an adenoviral packaging signal and non-viral, and non-coding stuffer nucleic acid sequences. In some embodiments, the expression of the human IL-1Ra gene is regulated by a NF-κB inducible promoter, which is located upstream of the reading frame of the nucleic acid sequence encoding the human IL-1Ra. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 95% homologous or identical to the nucleic acid sequence of SEQ ID NO: 2, SEQ ID NO: 3 or SEQ ID NO: 7. In some embodiments, the adenoviral-based biological delivery and expression system is isolated from a host cell that is infected with the helper-dependent adenoviral vector and a helper virus, wherein the adenoviral-based biological delivery and expression system comprises: a) 1.4×10⁸ to 1.4×10¹² GC of the helper-dependent adenoviral vector per milliliter (GC per ml); b) less than 15% helper virus particles; c) less than 10% empty capsids; d) not more than 100 μg/ml of host cell protein; e) not more than 20 ng/ml of host cell nucleic acid; f) not more than 35 EU/ml of endotoxin; and g) a Viral Particle to Infectious Unit Ratio of ≤than 300GC/TCID₅₀.

In some embodiments, the level of helper virus in the adenoviral-based biological delivery and expression system disclosed herein, is, or is less than, 15%, 14%, 13%, 12%, 11%, 10%, 9%, 8%, 7%, 6%, 5%, 4%, 3%, 2% or 1% helper virus particles, or a range defined by any two of the preceding values, for example, 1% to 2%, 2% to 3%, 3% to 4%, 4% to 5%, 5% to 6%, 6% to 7%, 7% to 8%, 8% to 9%, 9% to 10%, 10% to 11%, 11% to 12%, 12% to 13%, 13% to 14%, 14% to <15%, 1% to 15%, or 1% to 5% helper virus particles.

In some embodiments, the level of empty capsids in the adenoviral-based biological delivery and expression system disclosed herein, is, or is less than, 10%, 9%, 8%, 7%, 6%, 5%, 4%, 3%, 2% or 1% empty capsids, or a range defined by any two of the preceding values, for example, 1% to 2%, 2% to 3%, 3% to 4%, 4% to 5%, 5% to 6%, 6% to 7%, 7% to 8%, 8% to 9%, 9% to <10%, 1% to 10%, or 1% to 5% empty capsids. In some embodiments, the level of empty capsids and helper virus, in the adenoviral-based biological delivery and expression system disclosed herein, is the same.

As used herein, the terms “empty capsid,” and “empty particle,” have their ordinary and customary meaning as read in light of this disclosure, and refer to an adenoviral-based vector virion that includes a helper-dependent adenoviral protein shell but that lacks in whole or part the polynucleotide construct comprising a nucleic acid sequence encoding a human interleukin-1 receptor antagonist (IL-1Ra).

As used herein, the term “host cell” has its ordinary and customary meaning as read in light of this disclosure, and denotes, for example, microorganisms, yeast cells, insect cells, and mammalian cells, that can be, or have been, used as recipients of an a helper-dependent adenoviral vector construct of the present and a helper virus. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

In some embodiments, the adenoviral-based biological delivery and expression system of the present disclosure has a pH of 7.0±1.0. In some embodiments, the adenoviral-based biological delivery and expression system of the present disclosure has a pH of 6.0 to 6.5, 6.5 to 7.0, 7.0 to 7.5 or 7.0 to 8.0.

In some embodiments, the adenoviral-based biological delivery and expression system of the present disclosure has an osmolality of ≤600 mOsm/kg. In some embodiments, the adenoviral-based biological delivery and expression system of the present disclosure, has an osmolality of 100 mOsm/kg to 200 mOsm/kg, 200 mOsm/kg to 300 mOsm/kg, 300 mOsm/kg to 400 mOsm/kg, 400 mOsm/kg to 500 mOsm/kg, or 500 mOsm/kg to 600 mOsm/kg.

In some embodiments, the adenoviral-based biological delivery and expression system of the present disclosure comprises the helper-dependent adenoviral vector (HDAd) at: a) 1.4×10⁹ to 1.4×10¹²; b) 1.4×10⁹ to 1.4×10¹¹; or c) 1.4×10⁹ to 1.4×10¹⁰, GC per ml.

In some embodiments, the adenoviral-based biological delivery and expression system of the present disclosure comprises the helper-dependent adenoviral vector (HDAd) at a concentration of: ≥1.4×10⁹ GC per ml to <5.6×10⁹ GC per mL, 2.8×10⁹ GC per mL, ≥1.4×10¹⁰ GC per ml to <5.6×10¹⁰ GC per mL, 2.8×10¹⁰ GC per mL, ≥1.4×10¹¹ GC per ml to <5.6×10¹¹ GC per mL, 2.8×10¹¹ GC per mL, 1.4×10⁹ to 5.6×10⁹ GC per ml, 1.4×10¹⁰ to 5.6×10¹⁰ GC per ml, 1.4×10¹¹ to 5.6×10¹¹ GC per ml, 2×10⁹ to 5.6×10⁹ GC per ml, 2×10¹⁰ to 5.6×10¹⁰ GC per ml, 2×10¹¹ to 5.6×10¹¹ GC per ml, 2.8×10⁹ to 5.6×10⁹ GC per ml, 2.8×10¹⁰ to 5.6×10¹⁰ GC per ml, 2.8×10¹¹ to 5.6×10¹¹ GC per ml, at 2×10⁹ to 2.8×10⁹ GC per ml, 2×10¹⁰ to 2.8×10¹⁰ GC per ml, 2×10¹¹ to 2.8×10¹¹ GC per ml, 1.4×10⁹ to 2.8×10¹⁰ GC per ml, 1.4×10¹⁰ to 2.8×10¹¹ GC per ml, 1.4×10¹¹ to 2.8×10¹¹ GC per ml, 2.8×10⁹ to 1.4×10¹² GC per ml, 2.8×10¹⁰ to 1.4×10¹² GC per ml, 2.8×10¹¹ to 1.4×10¹² GC per ml, 2.8×10⁹ to 2.8×10¹¹ GC per ml, 2.8×10⁹ to 1.4×10¹⁰ GC per ml, 2.8×10¹⁰ to 2.8×10¹¹ GC per ml, 1.4×10⁹ GC per ml, 1.4×10¹⁰ GC per ml, 1.4×10¹¹ GC per ml, 1.4×10¹² GC per ml, 2×10⁹ GC per ml, 2×10¹⁰ GC per ml, 2×10¹¹ GC per ml, 2.8×10⁹ GC per ml, 2.8×10¹⁰ GC per ml, 2.8×10¹¹ GC per ml, 5.6×10⁹ GC per ml, 5.6×10¹⁰ GC per, or 5.6×10¹¹ GC per ml.

In some embodiments, the adenoviral-based biological delivery and expression system comprises a dose volume of 1 ml to 5 ml, 2 ml to 5 ml, 3 ml to 5 ml, 4 ml to 5 ml, 5 ml, or 2 ml. In some embodiments, the adenoviral-based biological delivery and expression system comprises a dose volume of 1 ml, 3 ml, or 4 ml. In some embodiments, the adenoviral-based biological delivery and expression system comprises a dose volume that is, is about, is less than, is less than about, is more than, is more than about, 0.1, 0.2, 0.3, 0.4, 0.5, 1, 2, 3, 4, or 5 ml, or a range defined by any two of the preceding values, for example, 0.1 ml to 5 ml, 0.5 ml to 5 ml, 0.5 ml to 2 ml, 1 ml to 5 ml, 2 ml to 5 ml, 4 ml to 5 ml, or 3 ml to 5 ml

In some embodiments, the adenoviral-based biological delivery and expression system of the present disclosure comprises the helper-dependent adenoviral vector (HDAd) at a total dose of: 7×10⁹ to 7×10¹² GC, 7×10⁹ to 7×10¹¹ GC, 7×10⁹ to 7×10¹⁰ GC, 7×10⁹ to 2.8×10¹⁰ GC, 7×10¹⁰ to 2.8×10¹¹ GC, 7×10¹¹ to 2.8×10¹² GC, 7×10¹⁰ to 7×10¹² GC, 7×10¹⁰ to 7×10¹¹ GC, 7×10¹¹ to 7×10¹² GC, 7×10⁹ to 2.8×10¹⁰ GC, 7×10¹⁰ to 2.8×10¹¹ GC, 7×10¹¹ to 2.8×10¹² GC, 10¹⁰ to 2.8×10¹⁰ GC, 10¹¹ to 2.8×10¹¹ GC, 10¹² to 2.8×10¹² GC, 2.8×10⁹ to 5.6×10⁹ GC, 2.8×10¹⁰ to 5.6×10¹⁰ GC, 2.8×10¹¹ to 5.6×10¹¹ GC, 10¹⁰ to 1.4×10¹⁰ GC, 10¹¹ to 1.4×10¹¹ GC, 10¹² to 1.4×10¹² GC, 7×10⁹ to 5.6×10¹¹ GC, 7×10¹⁰ to 5.6×10¹² GC, 7×10¹¹ to 5.6×10¹² GC, 1.4×10¹⁰ to 7×10¹² GC, 1.4×10¹¹ to 7×10¹² GC, 1.4×10¹² to 7×10¹² GC, 1.4×10¹⁰ to 1.4×10¹² GC, 1.4×10¹⁰ to 1.4×10¹¹ GC, 1.4×10¹¹ to 1.4×10¹² GC, 7×10⁹ GC, 7×10¹⁰ GC, 7×10¹¹ GC, 7×10¹² GC, 1.4×10¹⁰ GC, 1.4×10¹¹ GC, 1.4×10¹² GC, 2.8×10¹⁰ GC, 2.8×10¹¹ GC, or 2.8×10¹² GC. In some embodiments, the total dose is the amount injected into a single disc per administration.

### Pharmaceutical Compositions

In some embodiments, the adenoviral-based biological delivery and expression system of the present disclosure is incorporated into pharmaceutical compositions suitable for administration to intervertebral discs of a subject suffering from or developing DDD or a condition associated with DDD. In some embodiments, the subject is identified as suffering from or developing DDD or a condition associated with DDD. In some embodiments, the pharmaceutical composition of the present disclosure, is formulated for delivering the adenoviral-based biological delivery and expression system directly into the cells of one or more intervertebral discs of the subject in need thereof. In some embodiments, the pharmaceutical composition of the present disclosure, is formulated for delivering the adenoviral-based biological delivery and expression system directly into the cells of one or more degenerate discs or non-degenerate discs or both. In some embodiments, the pharmaceutical composition of the present disclosure, is formulated for delivering the adenoviral-based biological delivery and expression system directly into the cells of one or more degenerate discs. In some embodiments, the DDD as described herein is any one of cervical or lumbar degenerative disc disease. In some embodiments, the DDD is lumbar degenerative disc disease. In some embodiments, the pharmaceutical composition of the present disclosure, is formulated for delivering the adenoviral-based biological delivery and expression system directly into the cells of the intervertebral disc between the vertebral column bone pairs: C2-C3, C3-C4, C4-C5, C5-C6, C6-C7, C7-T1, T1-T2, T2-T3, T3-T4, T4-T5, T5-T6, T6-T7, T7-T8, T8-T9, T9-T10, T10-T11, T11-T12, T12-L1, L1-L2, L2-L3, L3-L4, L4-L5 and L5-S1, or any combination thereof. In some embodiments the subject has Facet Joint Syndrome (FJS). In some embodiments the subject does not have Facet Joint Syndrome (FJS).

In some embodiments, the pharmaceutical composition of the present disclosure, is formulated for delivering the adenoviral-based biological delivery and expression system into cells of the cartilaginous endplates (CEP), the highly organized annulus fibrosus (AF) and the central gelatinous nucleus pulposus (NP) region (nucleus pulposus (NP) cells) or a combination thereof, of the one or more intervertebral discs. In some embodiments, the pharmaceutical composition of the present disclosure, is formulated for delivering the adenoviral-based biological delivery and expression system into cells of the central gelatinous nucleus pulposus (NP) region (nucleus pulposus (NP) cells). In some embodiments, the pharmaceutical composition of the present disclosure, is formulated for delivering the adenoviral-based biological delivery and expression system into cells that are NP cells.

In some embodiments, the compositions of the present disclosure include pharmaceutical compositions comprising an adenoviral-based vector comprising a nucleic acid sequence encoding for human or mammalian interleukin-1 receptor antagonist (IL-1Ra), L ITR, R ITR, packaging signal and non-viral, non-coding stuffer nucleic acid sequences, wherein the expression of the human or mammalian interleukin-1 receptor antagonist (IL-1Ra) gene is regulated by an inflammation-sensitive promoter located upstream of the reading frame of the nucleic acid sequence encoding for the human or mammalian IL-1Ra. In some embodiments, the pharmaceutical composition is used for the treatment of DDD or a condition associated with DDD. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

Inflammation-sensitive promoters as used in the context of the present disclosure are promoters inducible by, for example, NF-κB, interleukin 6 (II-6), interleukin-1 (IL-1), tumor necrosis factor (TNF), cyclooxygenase 2 (COX-2), complement factor 3 (C3), serum amyloid A3 (SAA3), macrophage inflammatory protein-1a (MIP-1a), or hybrid constructs of the above. In some embodiments, the inflammation-sensitive promoter is an NF-κB5-ELAM promoter.

In some embodiments, the adenoviral-based vector (adenoviral-based biological delivery and expression system) of the compositions of the present disclosure, is a helper-dependent adenoviral-based vector (HDAd). In some embodiments, such compositions comprise the helper-dependent adenoviral vector viral particles as disclosed herein, and a pharmaceutically acceptable carrier. In some embodiments, the composition further comprises helper virus and/or empty viral particles in amounts disclosed herein. As used herein, the term “pharmaceutically acceptable carrier” has its ordinary and customary meaning as read in light of this disclosure, and is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference in its entirety. Non-limiting examples of such carriers or diluents include, but are not limited to, water, saline, ringer's solutions, dextrose solution, and 5% human serum albumin. In some embodiments, the liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. In some embodiments, supplementary active compounds are also incorporated into the compositions.

A pharmaceutical composition of the disclosure is formulated to be compatible with its intended route of administration, intradiscal injection into the intervertebral disc. Solutions or suspensions used for intradiscal injection can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The pharmaceutical preparation is enclosed in ampoules, disposable syringes or multiple dose vials made of glass or plastic. In some embodiments, the pharmaceutical composition is formulated for injecting directly into the intervertebral disc (intradiscal injection).

In some embodiments, the pharmaceutical composition of the disclosure, for injecting directly to the intervertebral disc further includes a corticosteroid, such as methylprednisolone, betamethasone, or triamcinolone, plus a small amount of a local anesthetic, such as lidocaine or bupivacaine. In some embodiments, the pharmaceutical composition of the disclosure for injecting directly to the intervertebral disc further includes a corticosteroid. In some embodiments, the corticosteroid is methylprednisolone, betamethasone, triamcinolone, or combinations thereof. In some embodiments, the corticosteroid, for example, methylprednisolone, betamethasone, triamcinolone, or combinations thereof, is in a separate dosage form from the adenoviral-based biological delivery and expression system, such that the corticosteroid can be administered before or after the adenoviral-based biological delivery and expression system. In some embodiments, the corticosteroid, for example, methylprednisolone, betamethasone, triamcinolone, or combinations thereof, is in the same dosage form (e.g., a single fluid) as the adenoviral-based biological delivery and expression system, such that the corticosteroid is administered simultaneously with the adenoviral-based biological delivery and expression system. In some embodiments, the pharmaceutical composition of the disclosure for injecting directly to the intervertebral disc further includes a local anesthetic. In some embodiments, the local anesthetic is lidocaine, bupivacaine or a combination thereof. In some embodiments, the local anesthetic, for example, lidocaine, bupivacaine or a combination thereof, is in a separate dosage form from the adenoviral-based biological delivery and expression system, such that the local anesthetic can be administered before or after the adenoviral-based biological delivery and expression system. In some embodiments, the local anesthetic, for example, lidocaine, bupivacaine or a combination thereof, is in the same dosage form (e.g., a single fluid) as the adenoviral-based biological delivery and expression system, such that the local anesthetic is administered simultaneously with the adenoviral-based biological delivery and expression system. In some embodiments, the pharmaceutical composition of the disclosure, for injecting directly to the intervertebral disc includes at least one viscosity enhancing agent. In some embodiments, the pharmaceutical composition of the disclosure, for injecting directly to the intervertebral disc includes at least one preservative. The viscosity enhancing agent can be any one or more viscosity enhancing agents known in the art, for examples as described in Handbook of Pharmaceutical Excipients Ninth edition, Paul J Sheskey, Bruno C Hancock, Gary P Moss, David J Goldfarb; and The United States pharmacopeia: USP 30; The National formulary: NF 25), which is herein incorporated by reference in its entirety.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and should be fluid to the extent that easy syringeability exists. It must be stable under the conditions of manufacture and storage and must be preserved against the contaminating action of microorganisms such as bacteria and fungi. In some embodiments, the carrier is a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

### Chemical Properties of the Adenoviral Expression and Delivery System of the Disclosure

In some embodiments, the capsid of the adenoviral-based biological delivery and expression system of the disclosure (e.g., FX201/PCRX-201) is unenveloped and comprises of 29.3 kb double-stranded DNA. The theoretical molecular weight of the capsid is 103.9 megadaltons (MDa) and the genome is 18.1 MDa. The capsid of FX201 can have a diameter of approximately 100 nm.

Formulations: In some embodiments, the adenoviral-based biological delivery and expression system of the disclosure (e.g., FX201/PCRX-201) is formulated in a buffer composed of about 1-20 mM, TRIS, about 50-100 mM NaCl, 0.01-1% weight/volume (w/v) Polysorbate 80, 1-10% (w/v) sucrose, 0.1-10 mM MgCl₂, 50-500 μM EDTA, 1-5% volume/volume (v/v) ethanol, and 5-50 mM L-histidine. In some embodiments, the adenoviral-based biological delivery and expression system of the disclosure (e.g., FX201) is formulated in a buffer composed of 10 mM TRIS, 75 mM NaCl, 0.02% (weight/volume (w/v) Polysorbate 80, 5% (w/v) sucrose, 1.0 mM MgCl₂, 100 μM EDTA, 0.5% (volume/volume (v/v) of ethanol), and 10 mM L-histidine. In some embodiments, the product is a clear to slightly opalescent, colorless suspension with no visible particulates.

Storage Conditions and Stability: In some embodiments, the adenoviral-based biological delivery and expression system of the disclosure (e.g., FX201/PCRX-201) is stored as a frozen liquid at ≤−65° C. In some embodiments, the adenoviral-based biological delivery and expression system of the disclosure (e.g., FX201) is stable for at least 3 months, at least 6 months or at least 12 months when stored at ≤−65° C. In some embodiments, the adenoviral-based biological delivery and expression system of the disclosure (e.g., FX201) is stable for at least 24 months when stored at ≤−65° C. In some embodiments, once thawed, the product should be stored at 2-8° C. and used within 7 days. In some embodiments, once thawed, the product should be stored at 2-8° C. and used within 14 days. In some embodiments, the adenoviral-based biological delivery and expression system of the disclosure (e.g., FX201) may be kept at room temperature (RT) for some period of time. In some embodiments, once a vial is ready for use it is held at RT in vial for no more than 7 hours (in some embodiments, vials held at RT cannot be returned to refrigeration for later use). In some embodiments, once a vial is ready for use it is held at RT in vial for no more than 12 hours (in some embodiments, vials held at RT cannot be returned to refrigeration for later use). In some embodiments, once the dosage is prepared in the syringe, it must be held at RT and used within 4 hours. In some embodiments, once the dosage is prepared in the syringe, it must be held at RT and used within 8 hours.

In some embodiments, the pharmaceutical compositions of the present disclosure comprising an adenoviral-based biological delivery and expression system (adenoviral-based vector) is used for the treatment of degenerated disc disease or a condition associated with DDD, wherein the adenoviral-based biological delivery and expression system comprises genome copies (GC) of an adenoviral-based vector comprising a nucleic acid sequence encoding a human interleukin-1 receptor antagonist (IL-1Ra); further comprising left and right inverted terminal repeats, an adenoviral packaging signal and non-viral, and non-coding stuffer nucleic acid sequences, optionally wherein the expression of the human IL-1Ra gene is regulated by an inflammation-sensitive promoter, which is located upstream of the reading frame of the nucleic acid sequence encoding the human IL-1Ra. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 95% homologous or identical to the nucleic acid sequence of SEQ ID NO: 2, SEQ ID NO: 3 or SEQ ID NO: 7. In some embodiments, the adenoviral-based biological delivery and expression system comprises 1.4×10⁸ to 1.4×10¹² GC of the adenoviral-based vector per milliliter (ml). In some embodiments, the inflammation-sensitive promoter is a promoter inducible by any one of NF-κB, interleukin 6 (II-6), interleukin-1 (IL-1), tumor necrosis factor (TNF), cyclooxygenase 2 (COX-2), complement factor 3 (C3), serum amyloid A3 (SAA3), macrophage inflammatory protein-1a (MIP-1a), or hybrid constructs of the above. In some embodiments, the inflammation-sensitive promoter is a NF-κB inducible promoter. In some embodiments, the NF-κB inducible promoter is an NF-κB5-ELAM promoter.

In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 96%, 97%, 98% or 99% homologous or identical to the nucleic acid sequence of SEQ ID NO: 2, SEQ ID NO: 3 or SEQ ID NO: 7. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 99% homologous or identical to the nucleic acid sequence of SEQ ID NO: 2, SEQ ID NO: 3 or SEQ ID NO: 7. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is of SEQ ID NO: 2, SEQ ID NO: 3 or SEQ ID NO: 7.

In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least 95% homologous or identical to the nucleic acid sequence of SEQ ID NO: 7. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences is at least at least 96%, 97%, 98% or 99% homologous or identical to the nucleic acid sequence of SEQ ID NO: 7.

In some embodiments, the nucleic acid sequence encoding the IL-1Ra comprises the nucleic acid of SEQ ID NO 1. In some embodiments, the nucleic acid sequence encoding the IL-1Ra comprises the nucleic acid of comprise the nucleic acid of SEQ ID NO 4. SEQ ID NO: 4 is a codon optimized version of the original coding sequence of human IL-1Ra (SEQ ID NO: 5), wherein the codon optimized sequence according to SEQ ID NO: 4 has: a) a codon adaptive index (CAI) of 0.96 compared a CAI of 0.78 in the wild type human IL-1Ra protein, b) 85% of the codons within the highest usage frequency, as compared to a highest usage frequency of 56% in the wild type human IL-1Ra protein, c) an average GC content of 60.4 as compared to an average GC content of 51.98 in the wild type human IL-1Ra protein, and d) no negative cis acting elements including: splice site (GGTAAG), splice site (GGTGAT), polyA (AATAAA), polyA (ATTAAA), destabilizing (ATTTA), polyT (TTTTTT) and polyA (AAAAAAA) as compared to the wild type human IL-1Ra protein.

In some embodiments, the amino acid sequence of the human IL-1Ra is according to SEQ ID NO: 6. In some embodiments, the nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences comprises, consists essentially of, or consists of the nucleic acid sequence of SEQ ID NO: 2, SEQ ID NO: 3 or SEQ ID NO: 7.

In some embodiments, the adenoviral-based vector additionally comprises a marker gene encoding a protein product that is visually or instrumentally detectable to monitor the presence of the vector sequences in infected cells. The marker gene can be a gene encoding any one of a fluorescent protein, an enzyme or a detectable cell surface protein. In some embodiments, the marker gene is a gene encoding any one of green fluorescent protein LacZ, or luciferase enzyme. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD.

In some embodiments, the pharmaceutical compositions of the present disclosure comprising the adenoviral-based biological delivery and expression system comprises: a) 1.4×10⁹ to 1.4×10¹²; b) 1.4×10⁹ to 1.4×10¹¹; or c) 1.4×10⁹ to 1.4×10¹⁰ genome copies (GC) of the adenoviral-based vector per ml of the pharmaceutical composition.

In some embodiments, the pharmaceutical compositions of the present disclosure comprising the adenoviral-based biological delivery and expression system comprises 1.0×10¹⁰ to 1.0×10¹², 1.0×10¹⁰ to 1.0×10¹¹, 1.0×10¹¹ to 1.0×10¹², 9×10¹⁰ to 9×10¹¹, 1.4×10¹⁰ to 1.4×10¹², 1.4×10¹⁰ to 1.4×10¹¹, 1.4×10¹¹ to 1.4×10¹², 1.4×10⁹ to 5.6×10⁹, 1.4×10¹⁰ to 5.6×10¹⁰, 1.4×10¹¹ to 5.6×10¹¹, 2×10⁹ to 5.6×10⁹, 2×10¹⁰ to 5.6×10¹⁰, 2×10¹¹ to 5.6×10¹¹, 2.8×10⁹ to 5.6×10⁹, 2.8×10¹⁰ to 5.6×10¹⁰, 2.8×10¹¹ to 5.6×10¹¹, 2×10⁹ to 2.8×10⁹, 2×10¹⁰ to 2.8×10¹⁰, 2×10¹¹ to 2.8×10¹¹, 1.4×10⁹ to 2.8×10¹⁰, 1.4×10¹⁰ to 2.8×10¹¹, 1.4×10¹¹ to 2.8×10¹¹, 2.8×10⁹ to 1.4×10¹², 2.8×10¹⁰ to 1.4×10¹², 2.8×10¹¹ to 1.4×10¹², 2.8×10⁹ to 2.8×10¹¹, 2.8×10⁹ to 1.4×10¹⁰, 2.8×10¹⁰ to 2.8×10¹¹, 1.4×10⁹, 1.4×10¹⁰, 1.4×10¹¹, 1.4×10¹², 2×10⁹, 2×10¹⁰, 2×10¹¹, 2.8×10⁹, 2.8×10¹⁰, 2.8×10¹¹, 5.6×10⁹, 5.6×10¹⁰, or 5.6×10¹¹ genome copies (GC) of the adenoviral-based vector per ml of the pharmaceutical composition. In some embodiments, the pharmaceutical compositions of the present disclosure comprising the adenoviral-based biological delivery and expression system comprises 1×10⁸ GC, 1×10⁹ GC, 1×10¹⁰ GC, 1×10¹¹ GC, 2×10¹¹ GC, 2×10⁹ GC, 2×10¹⁰ GC, 2×10¹¹ GC, 3×10⁸ GC, 3×10⁹ GC, 3×10¹⁰ GC, or 3×10¹¹ GC of the adenoviral-based vector per ml of the pharmaceutical composition, or a range defined by any two of the preceding values, for example 1×10⁸ GC to 3×10¹¹ GC, 1×10⁸ GC to 3×10⁹ GC, or 1×10⁹ GC to 3×10⁹ GC of the adenoviral-based vector per ml of the pharmaceutical composition.

In some embodiments, the pharmaceutical compositions of the present disclosure comprising the adenoviral-based biological delivery and expression system comprises a dose volume of 1 ml to 5 ml, 2 ml to 5 ml, 4 ml to 5 ml, 3 ml to 5 ml, or up to 5 ml. In some embodiments, the pharmaceutical compositions of the present disclosure comprising the adenoviral-based biological delivery and expression system comprises a dose volume that is, is about, is less than, is less than about, is more than, is more than about, 0.1, 0.2, 0.3, 0.4, 0.5, 1, 2, 3, 4, or 5 ml, or a range defined by any two of the preceding values, for example, 0.1 ml to 5 ml, 0.5 ml to 5 ml, 0.5 ml to 2 ml, 1 ml to 5 ml, 2 ml to 5 ml, 4 ml to 5 ml, or 3 ml to 5 ml.

In some embodiments, the pharmaceutical compositions of the present disclosure comprising the adenoviral-based biological delivery and expression system comprises a total dose of: 7×10⁹ to 7×10¹² GC, 7×10⁹ to 7×10¹¹ GC, 7×10⁹ to 7×10¹⁰ GC, 7×10¹⁰ to 7×10¹² GC, 7×10¹⁰ to 7×10¹¹ GC, 7×10¹¹ to 7×10¹² GC, 7×10⁹ to 2.8×10¹⁰ GC, 7×10¹⁰ to 2.8×10¹¹ GC, 7×10¹¹ to 2.8×10¹² GC, 7×10⁹ to 2.8×10¹¹ GC, 7×10⁹ to 2.8×10¹² GC, 7×10¹⁰ to 2.8×10¹² GC, 1×10¹⁰ to 2.8×10¹⁰ GC, 1×10¹¹ to 2.8×10¹¹ GC, 1×10¹² to 2.8×10¹² GC, f 2.8×10⁹ to 5.6×10⁹ GC, 2.8×10¹⁰ to 5.6×10¹⁰ GC, 2.8×10¹¹ to 5.6×10¹¹ GC, 1×10¹⁰ to 1.4×10¹⁰ GC, 1×10¹¹ to 1.4×10¹¹ GC, 1×10¹² to 1.4×10¹² GC, 7×10⁹ to 5.6×10¹¹ GC, 7×10¹⁰ to 5.6×10¹² GC, 7×10¹¹ to 5.6×10¹² GC, of 1.4×10¹⁰ to 7×10¹² GC, 1.4×10¹¹ to 7×10¹² GC, 1.4×10¹² to 7×10¹² GC, 1.4×10¹⁰ to 1.4×10¹² GC, 1.4×10¹⁰ to 1.4×10¹¹ GC, 1.4×10¹¹ to 1.4×10¹² GC, 7×10⁹ GC, 7×10¹⁰ GC, 7×10¹¹ GC, 7×10¹² GC, 1.4×10¹⁰ GC, 1.4×10¹¹ GC, 1.4×10¹² GC, 2.8×10¹⁰ GC, 2.8×10¹¹ GC, or 2.8×10¹² GC of the adenoviral-based vector. In some embodiments, the pharmaceutical compositions of the present disclosure comprising the adenoviral-based biological delivery and expression system comprises a total dose of: 1×10⁸ GC, 1×10⁹ GC, 1×10¹⁰ GC, 1×10¹¹ GC, 2×10⁸ GC, 2×10⁹ GC, 2×10¹⁰ GC, 2×10¹¹ GC, 3×10⁸ GC, 3×10⁹ GC, 3×10¹⁰ GC, or 3×10¹¹ GC, or a range defined by any two of the preceding values, for example 1×10⁸ GC to 3×10¹¹ GC, 1×10⁸ GC to 3×10⁹ GC, or 1×10⁹ GC to 3×10⁹ GC. In some embodiments of the pharmaceutical compositions of the present disclosure, the adenoviral-based vector of the adenoviral-based biological delivery and expression system is a helper-dependent adenoviral vector (HDAd).

In some embodiments, the pharmaceutical compositions of the present disclosure is formulated for intradiscal injection into the human intervertebral disc. In some embodiments, the composition is not injected intra-articularly. In some embodiments, the pharmaceutical compositions of the present disclosure is formulated for intradiscal injection into the intervertebral disc of a subject. In some embodiments, the pharmaceutical compositions of the present disclosure is formulated for intradiscal injection into the nucleus pulposus of the intervertebral disc. In some embodiments the subject has Facet Joint Syndrome (FJS). In some embodiments the subject does not have Facet Joint Syndrome (FJS).

In some embodiments, the pharmaceutical composition comprising an adenoviral-based biological delivery and expression system of the present disclosure comprises viral particles of a helper-dependent adenoviral vector quantified as either Genome copies (GC) of the adenoviral-based vector per milliliter (ml), or viral particles (VP) of the adenoviral-based vector per milliliter (ml), wherein the 1 VP/ml corresponds to 1.4 GC/ml.

In some embodiments, the pharmaceutical compositions of the present disclosure comprises an adenoviral-based biological delivery and expression system comprising 1.4×10⁸ to 1.4×10¹² genome copies of the adenoviral-based vector (GC) per milliliter (ml). In some embodiments, the pharmaceutical compositions can also comprise 10⁸ to 10¹² viral particles (VP) of the helper-dependent adenoviral vector of the disclosure, per milliliter (ml) of fluid in an intervertebral disc, or ml of volume of an intervertebral disc.

In some embodiments, the pharmaceutical compositions comprising the adenoviral-based biological delivery and expression system comprises 10⁹ to 10¹²; 10⁹ to 10¹¹; or 10⁹ to 10¹⁰ VP of the adenoviral-based vector per ml of fluid in an intervertebral disc, or ml of volume of an intervertebral disc. In some embodiments, the pharmaceutical compositions comprising the adenoviral-based biological delivery and expression system comprises 10⁹ to 10¹¹ VP of the adenoviral-based vector per ml of fluid in an intervertebral disc, or ml of volume of an intervertebral disc.

In some embodiments, the pharmaceutical compositions comprising the adenoviral-based biological delivery and expression system comprises 2.8×10⁹ to 2.8×10¹¹, 2.8×10⁹ to 2.8×10¹⁰, 2.8×10¹⁰ to 2.8×10¹¹, 2×10⁹ to 2×10¹¹, 2×10⁹ to 2×10¹⁰, or 2×10¹⁰ to 2×10¹¹ VP of the adenoviral-based vector per ml of fluid in an intervertebral disc, or ml of volume of an intervertebral disc. In some embodiments, the pharmaceutical compositions comprising the adenoviral-based biological delivery and expression system comprises 1×10⁸ VP, 1×10⁹ VP, 1×10¹⁰ VP, 1×10¹¹ VP, 2×10⁸ VP, 2×10⁹ VP, 2×10¹⁰ VP, 2×10¹¹ VP, 3×10⁸ VP, 3×10⁹ VP, 3×10¹⁰ VP, or 3×10¹¹ VP, or a range defined by any two of the preceding values, for example 1×10⁸ VP to 3×10¹¹ VP, 1×10⁸ VP to 3×10⁹ VP, or 1×10⁹ VP to 3×10⁹ VP of the adenoviral-based vector per ml of fluid in an intervertebral disc, or ml of volume of an intervertebral disc.

In some embodiments, the pharmaceutical compositions comprising the adenoviral-based biological delivery and expression system comprises 2.8×10⁹, 2.8×10¹⁰, 2.8×10¹¹, or 2.8×10¹¹ VP of the adenoviral-based vector per ml of fluid in an intervertebral disc, or ml of volume of an intervertebral disc.

In some embodiments, the pharmaceutical compositions comprising the adenoviral-based biological delivery and expression system comprises 10⁹ to 10¹²; 10⁹ to 10¹¹; or 10⁹ to 10¹⁰ VP of the adenoviral-based vector per ml of fluid in an intervertebral disc, or ml of volume of an intervertebral disc. In some embodiments, the pharmaceutical compositions comprising the adenoviral-based biological delivery and expression system comprises 10⁹ to 10¹¹ VP of the adenoviral-based vector per ml of fluid in an intervertebral disc, or ml of volume of an intervertebral disc.

In some embodiments, the method of infecting cells of one or more intervertebral discs of a human suffering from DDD or a condition associated with DDD, with an adenoviral-based biological delivery and expression system, of the present disclosure comprises infecting the one or more intervertebral discs of the human in need thereof with 10⁸ to 10¹² viral particles (VP) of the adenoviral-based vector of the disclosure, per milliliter (ml) of fluid in an intervertebral disc, or ml of volume of an intervertebral disc.

In some embodiments, the pharmaceutical compositions comprising the adenoviral-based biological delivery and expression system comprises 10⁹ to 10¹²; 10⁹ to 10¹¹; or 10⁹ to 10¹⁰ VP of the adenoviral-based vector per ml of fluid in an intervertebral disc, or ml of volume of an intervertebral disc. In some embodiments, the pharmaceutical compositions comprising the adenoviral-based biological delivery and expression system comprises 10⁹ to 10¹¹ VP of the adenoviral-based vector per ml of fluid in an intervertebral disc, or ml of volume of an intervertebral disc.

In some embodiments, the intervertebral disc contains about 0.5 ml to about 20 ml, 0.5 ml to 10 ml, 0.5 ml to 5 ml, 5 ml to 10 ml, 10 ml to 15 ml, 15 ml to 17 ml, or 17 ml to 20 ml of fluid or ml of volume of an intervertebral disc.

### Methods of the Present Disclosure

In some embodiments, the present disclosure provides a method of infecting cells of one or more intervertebral discs of a subject suffering from, or identified as suffering from, degenerative disc disease (DDD), with an adenoviral-based biological delivery and expression system, wherein the method comprises the steps of: a) infecting cells of one or more intervertebral discs of the subject, or the subject in need thereof, with the pharmaceutical composition comprising an amount, optionally an effective amount, of an adenoviral-based biological delivery and expression system of the present disclosure; and b) expressing IL-1Ra in the cells of the one or more intervertebral discs. In some embodiments, the adenoviral-based biological delivery and expression system further comprises a nucleic acid encoding one or more proteins in addition to interleukin-1 receptor antagonist (IL-1Ra) protein. In some embodiments the additional protein is a therapeutic protein for treating DDD or a or a condition associated with DDD. In some embodiments, the method further comprises expressing the additional protein in the cells of the one or more intervertebral discs. In some embodiments the subject has Facet Joint Syndrome (FJS). In some embodiments the subject does not have Facet Joint Syndrome (FJS).

In some embodiments, the method further comprises administering a corticosteroid, for example methylprednisolone, betamethasone, triamcinolone, or combinations thereof, into the intervertebral disc of a subject. In some embodiments, the corticosteroid is formulated in a single pharmaceutical composition with the adenoviral-based biological delivery and expression system such that the corticosteroid is administered simultaneously with the adenoviral-based biological delivery and expression system. In some embodiments, the corticosteroid is in a separate dosage form from the adenoviral-based biological delivery and expression system, such that the corticosteroid can be administered prior to or after the administration of the adenoviral-based biological delivery and expression system to an intervertebral disc of a subject. In some embodiments, the method further comprises administering a local anesthetic, for example lidocaine, bupivacaine or a combination thereof, into the intervertebral disc of a subject. In some embodiments, the local anesthetic is formulated in a single pharmaceutical composition with the adenoviral-based biological delivery and expression system such that the local anesthetic is administered simultaneously with the adenoviral-based biological delivery and expression system. In some embodiments, the local anesthetic is in a separate dosage form from the adenoviral-based biological delivery and expression system, such that the local anesthetic can be administered prior to or after the administration of the adenoviral-based biological delivery and expression system to an intervertebral disc of a subject. In some embodiments, the method further comprises administering both a corticosteroid and a local anesthetic to an intervertebral disc of a subject. In some embodiments, one or both of the corticosteroid and the local anesthetic are formulated in a single pharmaceutical composition with the adenoviral-based biological delivery and expression system such that the corticosteroid and/or local anesthetic are administered simultaneously with the adenoviral-based biological delivery and expression system. In some embodiments, one or both of the corticosteroid and local anesthetic are in separate dosage form(s) from the adenoviral-based biological delivery and expression system, such that the corticosteroid and/or local anesthetic can be administered prior to or after the administration of the adenoviral-based biological delivery and expression system to an intervertebral disc of a subject.

In some embodiments, the method further comprises administering a fluid into the intervertebral disc of a subject after administration of the adenoviral-based biological delivery and expression system. In some embodiments, the fluid is a saline solution, for example a 0.9% saline solution. In some embodiments the fluid is a buffered solution, for example PBS. In some embodiments, the volume of the fluid administered into the intervertebral disc of a subject after administration of the adenoviral-based biological delivery and expression system is, is about, is less than, is less than about, is more than, is more than about, 25, 30, 35, 40, 45, 50, 55, 60, 65, 70, 75, 80, 85, 90, 95, 100, 150, 200, 250, 300, 350, 400, 450, 500, 600, 700, 800, 900, 1000 μl, or a range defined by any two of the preceding values, for example 25-1000, 25-500, 25-200, 50-150, 500-1000, 200-800, 200-600, 400-800, or 600-800 μl. In some embodiments, the administration of the fluid comprises intradiscal injection or infusion of the fluid. In some embodiments, the administration of the fluid increases the distribution of the adenoviral-based biological delivery and expression system in the NP of the disc.

In some embodiments, the cells of the one or more intervertebral discs are infected once with the adenoviral-based biological delivery and expression system. In some embodiments, the cells of the one or more intervertebral discs are infected two or more times with the adenoviral-based biological delivery and expression system. In some embodiments, the cells are infected by intradiscal injection of the pharmaceutical composition.

In some embodiments, when the cells of the one or more intervertebral discs are infected two or more times with an adenoviral-based biological delivery and expression system, each infection comprises a different number of genome copies of the adenoviral-based vector. In some embodiments, when the one or more intervertebral discs are infected at least twice with an adenoviral-based biological delivery and expression system, the first infection comprises a number of GC per ml that is less than the number of GC per ml of the second or any subsequent infection. In some embodiments, when the one or more intervertebral discs are infected at least twice with an adenoviral-based biological delivery and expression system, the first infection comprises a number of GC per ml that is more than the number of GC per ml of the second or any subsequent infection.

In some embodiments, when the cells of the one or more intervertebral discs are infected two or more times with an adenoviral-based biological delivery and expression system, wherein each infection comprises a different number of genome copies of the adenoviral-based vector, the first infection comprises 1.4×10⁹ GC per ml to 1.4×10¹⁰ GC/ml, and the second or subsequent infection comprises 1.4×10¹¹ to 1.4×10¹² GC per ml, the first infection comprises 1.4×10¹⁰ to 1.4×10¹¹ GC/ml, and the second or subsequent infection comprises 1.4×10¹¹ to 1.4×10¹² GC per ml, the first infection comprises 1.4×10⁹ to 1.4×10¹⁰ GC/ml, and the second or subsequent infection comprises 1.4×10¹⁰ to 1.4×10¹¹ GC per ml, the first infection comprises 1.4×10⁹ GC per ml to 5.6×10⁹ GC/ml, and the second or subsequent infection comprises 1.4×10¹⁰ to 5.6×10¹⁰ GC per ml, the first infection comprises 1.4×10¹⁰ to 5.6×10¹⁰ GC/ml, and the second or subsequent infection comprises 1.4×10¹¹ to 5.6×10¹¹ GC per ml, the first infection comprises 1.4×10⁹ to 5.6×10⁹ GC/ml, and the second or subsequent infection comprises 1.4×10¹¹ to 5.6×10¹¹ GC per ml, the first infection comprises 2.8×10⁹ GC per ml and the second or subsequent infection comprises 2.8×10¹⁰ GC per ml, the first infection comprises 2.8×10¹⁰ GC per ml and the second or subsequent infection comprises 2.8×10¹¹ GC per ml, the first infection comprises 2.8×10⁹ GC per ml and the second or subsequent infection comprises 2.8×10¹¹ GC per ml, the first infection comprises a number of GC per ml that is more than the number of GC per ml of the second or any subsequent infection, the first infection comprises 1.4×10¹¹ to 1.4×10¹² GC per ml, and the second or subsequent infection comprises 1.4×10⁹ GC per ml to 1.4×10¹⁰ GC/ml, the first infection comprises 1.4×10¹¹ to 1.4×10¹² GC per ml, and the second or subsequent infection comprises 1.4×10¹⁰ to 1.4×10¹¹ GC/ml, the first infection comprises 1.4×10¹⁰ to 1.4×10¹¹ GC per ml, and the second or subsequent infection comprises 1.4×10⁹ to 1.4×10¹⁰ GC/ml, the first infection comprises 1.4×10¹⁰ to 5.6×10¹⁰ GC per ml, and the second or subsequent infection comprises 1.4×10⁹ GC per ml to 5.6×10⁹ GC/ml, the first infection comprises 1.4×10¹¹ to 5.6×10¹¹ GC per ml, and the second or subsequent infection comprises 1.4×10¹⁰ to 5.6×10¹⁰ GC/ml, the first infection comprises 1.4×10¹¹ to 5.6×10¹¹ GC per ml, and the second or subsequent infection comprises 1.4×10⁹ to 5.6×10⁹ GC/ml, the first infection comprises 2.8×10¹⁰ GC per ml and the second or subsequent infection comprises 2.8×10⁹ GC per ml, the first infection comprises 2.8×10¹¹ GC per ml and the second or subsequent infection comprises 2.8×10¹⁰ GC per ml, or the first infection comprises 2.8×10¹¹ GC per ml and the second or subsequent infection comprises 2.8×10⁹ GC per ml.

In some embodiments, when the cells of the one or more intervertebral discs are infected two or more times with an adenoviral-based biological delivery and expression system, each infection comprises the same number of genome copies of the adenoviral-based vector.

In some embodiments, when the cells of the one or more intervertebral discs are infected at least twice with an adenoviral-based biological delivery and expression system, each infection comprises 1.4×10⁹ to 5.6×10⁹, 1.4×10¹⁰ to 5.6×10¹⁰, 1.4×10¹¹ to 5.6×10¹¹, 2.8×10⁹, 2.8×10¹⁰, or 2.8×10¹¹ GC per ml.

In some embodiments, when the cells of the one or more intervertebral discs are infected two or more times with an adenoviral-based biological delivery and expression system, each infection is done in the same intervertebral disc of the subject.

In some embodiments, when the cells of the one or more intervertebral discs are infected two or more times with an adenoviral-based biological delivery and expression system, every second and subsequent infection is done in an intervertebral disc of the subject that is different than the intervertebral disc in which the previous infection was done.

In some embodiments, the infecting of the one or more intervertebral discs comprises intradiscal injection of a pharmaceutical composition of the present disclosure. The “infecting of the one or more intervertebral discs” as described herein has its ordinary and customary meaning as read in light of this disclosure, and includes administering the pharmaceutical composition of the present disclosure to the one or more intervertebral discs affected by DDD or a condition associated with DDD, wherein the administering comprises injecting the pharmaceutical composition into the one or more intervertebral discs affected by affected by DDD or a condition associated with DDD intradiscally into the one or more intervertebral discs. In some embodiments, the administering of the pharmaceutical composition of the present disclosure to the intervertebral discs affected by DDD or a condition associated with DDD, is done by injecting the pharmaceutical composition into the one or more intervertebral discs affected by DDD or a condition associated with DDD, by intradiscal injection. In some embodiments, the administering of the pharmaceutical composition of the present disclosure to the intervertebral discs affected by DDD or a condition associated with DDD, is done by injecting the pharmaceutical composition into the one or more intervertebral discs affected by DDD or a condition associated with DDD, by a method that is not intra-articularly.

In some embodiments, the infecting of the cells of the one or more intervertebral discs comprises injecting the pharmaceutical composition of the present disclosure into the cartilaginous endplates (CEP) region, the highly organized annulus fibrosus (AF) region or the central gelatinous nucleus pulposus (NP) region (nucleus pulposus (NP) cells) or a combination thereof, of the one or more intervertebral discs. In some embodiments, the infecting of the cells of the one or more intervertebral discs comprises injecting the pharmaceutical composition into the nucleus pulposus (NP) region of the one or more intervertebral discs.

### Treatment Monitoring

In some embodiments, the methods of the present disclosure can further comprise the step of: c) monitoring the treatment or progress of DDD in the degenerated intervertebral discs of the subject following the expression of the IL-1Ra in the cells of the one or more intervertebral discs of the subject in need thereof, infected with the pharmaceutical composition comprising and amount, optionally an effective amount, of an adenoviral-based biological delivery and expression system of the present disclosure.

In some embodiments, the subject is a mammal. In some embodiments, the subject is a human. In some embodiments the subject has Facet Joint Syndrome (FJS). In some embodiments the subject does not have Facet Joint Syndrome (FJS).

In some embodiments, the monitoring of the treatment or progress of DDD comprises determining the level of NGF, NT-3, VEGF, Substance P, cytokines: IL-1β, IL-6, IL-8, TNF α, MMP 3, MMP 13, ADAMTS 4, aggrecan or collagen type II or a combination thereof, one or more intervertebral discs of the subject in need thereof, infected with the pharmaceutical composition comprising an amount, optionally an effective amount, of an adenoviral-based biological delivery and expression system of the present disclosure.

In some embodiments, the monitoring of the treatment or progress of DDD or a condition associated with DDD, in the one or more intervertebral discs of the subject in need thereof, infected with the pharmaceutical composition comprising an amount, optionally an effective amount, of an adenoviral-based biological delivery and expression system of the present disclosure, comprises: determining pain, physical function, patient global assessment, and intervertebral disc imaging of the subject in need thereof; evaluating progress of degenerated disc disease using scores from patient-reported pain and/or function measurements; or evaluating DDD associated pain by determining the visual analog scale (VAS) score of the subject, optionally the VAS is a questionnaire based scoring point scale system, as follows: 0 (no pain); 1-3 (mild pain); 4-6 (moderate to severe pain); 7-9 (very severe pain); and 10 (worst possible pain). In some embodiments, the monitoring of the treatment or progress of DDD or a condition associated with DDD, if the VAS score of the subject is higher or unchanged post-injection with the pharmaceutical composition of the present disclosure, the degenerative disc disease in the intervertebral disc of the subject is considered not managed or not treated. In some embodiments, the monitoring of the treatment or progress of DDD or a condition associated with DDD, if the VAS score of the subject is lower post-injection with the pharmaceutical composition of the present disclosure, the degenerative disc disease in the intervertebral disc of the subject is considered managed or treated.

In some embodiments, the monitoring of the treatment or progress of degenerated disc disease (DDD) or a condition associated with DDD, in the one or more intervertebral discs of the subject in need thereof, comprises evaluating loss or gain of function by Oswestry disability index (ODI), as follows: 0% to 20% (minimal disability); 21%-40% (moderate disability); 41%-60% (severe disability); 61%-80% (crippled): 81%-100% (bed-bound or exaggerating symptoms). In some embodiments, the monitoring of the treatment or progress of DDD or a condition associated with DDD, if the ODI score of the subject is higher or unchanged, post-injection with the pharmaceutical composition of the present disclosure, the degenerative disc disease in the intervertebral disc of the subject is considered not managed or not treated. In some embodiments, the monitoring of the treatment or progress of DDD or a condition associated with DDD, if the ODI score of the subject is lower, post-injection with the pharmaceutical composition of the present disclosure, the degenerative disc disease in the intervertebral disc of the subject is considered managed or treated.

In some embodiments, the monitoring of the treatment or progress of DDD or a condition associated with DDD, in the one or more one or more intervertebral discs of the subject in need thereof comprises physically examining the subject in need thereof, for any one or all of pain or tenderness in the neck or lower back, spine's flexibility and range of motion, pain and stiffness affecting movement (including sitting and walking), tingling, numbness, or weakness in the arms or legs. In some embodiments, the monitoring of the treatment or progress of DDD or a condition associated with DDD, in the one or more intervertebral discs of the subject in need thereof, comprises physically examining the human in need thereof for depression, sleep deprivation, hyperalgesia, central sensitization, and catastrophization or a combination thereof.

In some embodiments, the monitoring of the treatment or progress of DDD or a condition associated with DDD in the intervertebral discs of the subject in need thereof comprises using radiograph imaging to determine: (a) space between the vertebral bodies indicating change in height of the intervertebral discs; and (b) osteophytes formation and if the adjacent vertebral body endplates are sclerotic and irregular. In some embodiments, the monitoring of the treatment or progress of DDD or a condition associated with DDD in the intervertebral discs of the subject in need thereof comprises imaging the intervertebral discs of the subject in need thereof using any one or a combination of magnetic resonance imaging (MRI), ultrasound (US), and optical coherence tomography (OCT).

In some embodiments, the monitoring of the treatment or progress of DDD or a condition associated with DDD in the intervertebral discs of the subject in need thereof comprises: measurement of Interleukin-1 receptor antagonist (IL-1Ra) and Interleukin-1 beta (IL-1β) protein concentrations in the intervertebral discs; evaluating the immunological response to the adenoviral-based vector of the present disclosure; testing blood samples of the human treated with the pharmaceutical composition or method of the present disclosure, for the presence of anti-Capsid and anti-IL-1Ra antibodies; and/or testing IL-1Ra and IL-1β protein concentrations in intervertebral disc tissue and fluid samples of the human treated with the pharmaceutical composition or method of the present disclosure.

In some embodiments, the monitoring of the treatment or progress of DDD or a condition associated with DDD in the intervertebral discs of the subject in need thereof comprises determining the level NGF, NT-3, VEGF, Substance P, cytokines: IL-1β, IL-6, IL-8, TNF α, MMP 3, MMP 13, and ADAMTS 4, aggrecan or collagen type II or a combination thereof, in intervertebral disc tissue and fluid samples of the human treated with the pharmaceutical composition or method of the present disclosure. In some embodiments, the monitoring of the treatment or progress of DDD or a condition associated with DDD, if the level of any one or more of NGF, NT-3, VEGF, Substance P, cytokines: IL-1β, IL-6, IL-8, TNF α, MMP 3, MMP 13 and ADAMTS 4 is higher or unchanged post-injection with the pharmaceutical composition of the present disclosure, the degenerative disc disease in the intervertebral disc of the subject is considered not managed or not treated. In some embodiments, the monitoring of the treatment or progress of DDD or a condition associated with DDD, if the level of any one or more of aggrecan or collagen type II is lower post-injection with the pharmaceutical composition of the present disclosure, the degenerative disc disease in the intervertebral disc of the subject is considered not managed or not treated. In some embodiments, the monitoring of the treatment or progress of DDD or a condition associated with DDD: (a) increase in level of NGF, NT-3, VEGF, Substance P, IL-1β, IL-6, IL-8, TNF α, MMP 3, MMP 13 or ADAMTS 4 or a combination thereof, or (b) decrease or no change in the level of aggrecan or collagen type II or a combination thereof, or (c) both, in the one or more intervertebral discs post-injection with the pharmaceutical composition indicates that the degenerative disc disease in the Intervertebral disc of the subject is not managed or not treated. In some embodiments, the monitoring of the treatment or progress of DDD or a condition associated with DDD, if the level of any one or more of NGF, NT-3, VEGF, Substance P, cytokines: IL-1β, IL-6, IL-8, TNF α, MMP 3, MMP 13 and ADAMTS 4 is lower post-injection with the pharmaceutical composition of the present disclosure, the degenerative disc disease in the intervertebral disc of the subject is considered managed or treated. In some embodiments, the monitoring of the treatment or progress of DDD or a condition associated with DDD, if the level of any one or more of aggrecan or collagen type II is higher after infecting with the pharmaceutical composition of the present disclosure, post-injection with the pharmaceutical composition of the present disclosure, the degenerative disc disease in the intervertebral disc of the subject is considered managed or treated. In some embodiments, the monitoring of the treatment or progress of DDD: (a) decrease or no change in level of NGF, NT-3, VEGF, Substance P, IL-1β, IL-6, IL-8, TNF α, MMP 3, MMP 13 or ADAMTS 4 or a combination thereof, or (b) increase in the level of aggrecan or collagen type II or a combination thereof, or (c) both, in the one or more intervertebral discs post-injection with the pharmaceutical composition indicates that the degenerative disc disease in the Intervertebral disc of the subject is managed or treated

In some embodiments, the monitoring of the treatment or progress of DDD, is done by determining change in score based on histopathology scoring system for human intervertebral disc degeneration for the one or more intervertebral discs post-injection with the pharmaceutical composition. In some embodiments, the histopathology scoring system for human intervertebral disc degeneration is any one of the scoring systems known in the art, for example grading method described in Christine L. Le Maitre et al., JOR Spine, Vol. 4, Issue 2, June 2021 (Grade 0-3), Nachemson classification system (Grades 1-4), Thompson classification system (Grades 1-5), Gries classification system (Grades 1-4), Boos classification system (Grades 0-22), Sive classification system (Grades 0-12) and Rutges classification system (Grades 0-12). In some embodiments, the monitoring of the treatment or progress of DDD, an increase or no change in score based on histopathology scoring system for human intervertebral disc degeneration for the one or more intervertebral discs post-injection with the pharmaceutical composition, indicates that the degenerative disc disease in the intervertebral disc of the subject is not managed or treated. In some embodiments, the monitoring of the treatment or progress of DDD, a decrease in score based on histopathology scoring system for human intervertebral disc degeneration for the one or more intervertebral discs post-injection with the pharmaceutical composition, indicates that the degenerative disc disease in the intervertebral disc of the subject is managed or treated.

In some embodiments, the method of the present disclosure can further comprise the steps of: (d) continuing to administer the same effective amount of the adenoviral-based biological delivery and expression system to the cells of the one or more intervertebral discs of (a), if monitoring of (c) shows that the degenerative disc disease in the intervertebral disc of the subject is not managed or not treated; or (e) further adjusting the amount of the adenoviral-based biological delivery and expression system and administering to cells of the one or more intervertebral discs of (a), if monitoring of (c) shows that the degenerative disc disease in the intervertebral disc of the subject has progressed.

DDD, Condition Associated with DDD and Subjects Suffering Therefrom,

Low back pain (LBP) is the biggest cause of morbidity worldwide, affecting 80% of the population at some point in their lives (Hoy D G et al., Ann Rheum Dis. 2014; Hoy D et al., Ann Rheum Dis. 2014). Forty percent of LBP cases are attributable to failure of the intervertebral discs (IVDs) in a degenerative process known as degenerative disc disease (DDD) (Luoma K, et al., Spine. 2000) although disc degeneration per se is not always associated with LBP (Brinjikji W et al., AJNR Am J Neuroradiol. 2015). Patients suffering from chronic LBP due to IVD degeneration (IDD) or DDD whom have exhausted conservative treatment (e.g. pain relief medication and physiotherapy) have no remaining options other than invasive and costly surgical intervention. To date, there are no treatments that can halt or reverse DDD, despite the profound socioeconomic burden and impact of DDD, decreasing the quality of life of millions of people.

The normal IVD is composed of three morphologically distinct regions: the cartilaginous endplates (CEP), the highly organized annulus fibrosus (AF) and the central gelatinous nucleus pulposus (NP), which operate collectively to transfer loads and disperse energy evenly throughout the spine. The interactions among these three tissues also control local biophysical phenomena necessary to keep the IVD healthy. The CEP is a thin layer of hyaline cartilage that facilitates diffusion of nutrients and oxygen to the avascular internal structures of the IVD (Benneker L M et al., Spine. 2005; Bibby S R et al., Eur Spine J. 2004). The AF contains large amounts of fibrous collagen type I orientated into lamellae. It provides resistance to tensile forces from bending and twisting of the spine (Shankar H et al., Techniques in Regional Anesthesia and Pain Management. 2009) and to the lateral expansion of the highly hydrated NP under the effect of compressive loads. The NP extracellular matrix (ECM) is mainly composed of randomly arranged collagen type II fibers, in a matrix rich in proteoglycans (mainly aggrecan) (Mwale F et al., Eur. Cell Mater. 2004) that provide disc swelling capacity and resistance to compression while tissue swelling stretches the fiber of the collagen network (Dolan P et al., Clin. Biomech. (Bristol., Avon.). 2001). The cells of the disc are mechano-sensitive and this internal functional distribution of the mechanical loads locally generates important biophysical signals for the proper biological maintenance of the disc tissues.

DDD is characterized by progressive changes in the ECM due to altered cellular metabolism, matrix synthesis and an increase in degradation of normal matrix components, and changes in the composition of the matrix (Le Maitre C L et al., Biochem Soc Trans. 2007; Inkinen R I et al., J Rheumatol. 1998). Concurrently matrix degradation is accelerated by the upregulation of MMPs (matrix metalloproteinases) and ADAMTS (a disintegrin and metalloproteinase with thrombospondin motif(s) (Pockert A J et al., Arthritis Rheum. 2009; Le Maitre C L et al., J Pathol. 2004). Compositional changes in the matrix during IVD degeneration is also accompanied by cellular changes with increased apoptosis (Gruber H E et al., Spine. 1998, Zhao C Q et al., Apoptosis. 2006) and senescence (Heathfield S K et al., Arthritis Res. Ther. 2008; Gruber H E et al., Spine. 2007; Le Maitre C L et al., Arthritis Res Ther. 2007; Roberts S et al., Eur Spine J. 2006), together with decreased tissue cellularity (Vo N V et al., Orthop Res. 2016). The cells of the IVD produce a plethora of catabolic cytokines and chemokines (Vo N V et al., Orthop Res. 2016; Phillips K L et al., Osteoarthritis Cartilage. 2015; Phillips K L et al., Arthritis Res Ther. 2013; Shamji M F et al., Arthritis Rheum. 2010) with highest expression seen in the NP and inner AF (Phillips K L et al., Osteoarthritis Cartilage. 2015; Le Maitre C L et al., Arthritis Res Ther. 2005). IL-1 and IL-1Ra (IL-1 receptor antagonist) play pivotal role in pathogenesis of DDD (Hoyland J A et al., Rheumatology (Oxford). 2008; Phillips K L et al., Ann Rheum Dis. 2013; Karppinen J et al., Eur Spine J. 2009; Solovieva S et al., Eur Spine J. 2005; Solovieva S et al., Epidemiology. 2004; Solovieva S, et al., Pain. 2004; Eskola P J et al., Int J Mol Epidemiol Genet. 2012; Kim D H et al., Spine (Phila Pa 1976). 2010; Paz Aparicio J et al., Eur Spine J. 2011; Le Maitre C L et al., nt J Exp Pathol. 2006).

In some embodiments of the methods of the present disclosure, the subject suffering from, or identified as suffering from, DDD or a condition associated with DDD is a mammal. In some embodiments, the subject suffering from, or identified as suffering from, DDD or a condition associated with DDD is a human. In some embodiments the subject has Facet Joint Syndrome (FJS). In some embodiments the subject does not have Facet Joint Syndrome (FJS).

In some embodiments, the human suffering from, or identified as suffering from, DDD or a condition associated with DDD is more than 20 to more than 80 years of age, between 40-50 years of age, or more than 50 years of age. In some embodiments, the human suffering from, or identified as suffering from, DDD or a condition associated with DDD is -suffering from cervical disc disease: degeneration of discs occurs in the neck region of the spine, thoracic disc disease: degeneration of discs occurs in the mid-back of the spine, or lumbar disc disease: degeneration of discs occurs in the lower back of the spine. In some embodiments, the DDD is caused by degenerative processes including but not limited to dry out of the intervertebral disc or cracks in the intervertebral disc or intervertebral slip disc or intervertebral disc herniation. In some embodiments, the degenerative processes is due to an injury or aging.

In some embodiments, the condition associated with DDD is lower back pain, decreased back muscle tone, reduced flexibility of the back or blood clot or a combination thereof. In some embodiments, the condition associated with DDD is idiopathic low-back pain, lumbar radiculopathy, myelopathy, lumbar stenosis, spinal stenosis, osteoarthritis in the spine, spondylosis, spondylolisthesis, scoliosis, zygapophydeal joint degeneration or a combination thereof. In some embodiments the subject has Facet Joint Syndrome (FJS). In some embodiments the subject does not have Facet Joint Syndrome (FJS).

In some embodiments, the DDD is any one of grades 2, 3, 4 and 5, according to Pfirrmann classification, as described below: grade 2, nonhomogeneous shape with horizontal bands, some blurring between nucleus and annulus; grade 3, nonhomogeneous shape with blurring between nucleus and annulus, annulus shape still recognizable; grade 4, nonhomogeneous shape with hypointensity, annulus shape not intact and distinction between nucleus and annulus impossible, disc height usually decreased; grade 5, same as grade 4 but with collapsed disc space.

In some embodiments, the DDD is with annulus fibrosis of any one of Grades 1, 2, 3 and 4, as described below: Grade 1: the contrast medium passes into the cartilage endplate through a tear; Grade 2: the contrast medium flows into the bony endplate; Grade 3: the contrast medium enters into the cancellous bone of vertebra under endplate; Grade 4: the contrast medium leaks completely in the cancellous bone. In some embodiments, the different stages or grades of DDD is diagnosed using CT scan, MRI or x-ray or a combination thereof.

In some embodiments, the DDD as described herein is any one of cervical or lumbar degenerative disc disease. In some embodiments, the DDD is lumbar degenerative disc disease. In some embodiments, the DDD as described herein, is DDD of the intervertebral disc between the vertebral column bone pairs: C2-C3, C3-C4, C4-C5, C5-C6, C6-C7, C7-T1, T1-T2, T2-T3, T3-T4, T4-T5, T5-T6, T6-T7, T7-T8, T8-T9, T9-T10, T10-T11, T11-T12, T12-L1, L1-L2, L2-L3, L3-L4, L4-L5 and L5-S1, or any combination thereof. In some embodiments, the DDD is DDD of the intervertebral disc between the vertebral column bone pairs: L4-L5 or L5-S1 or a combination thereof.

In some embodiments, the human is suffering from, or identified as suffering from, DDD or a condition associated with DDD caused by ageing, genetic predisposition, obesity, metabolic diseases, joint injuries, repeated stress on the vertebral column, sports injury or bone deformities or a combination thereof. In some embodiments the subject has Facet Joint Syndrome (FJS). In some embodiments the subject does not have Facet Joint Syndrome (FJS).

In some embodiments, the human is suffering from, or identified as suffering from, DDD or a condition associated with DDD of any one of the following stages: Stage 1: The spine loses its normal balance, with possible damage to spinal curvature which in turn affects posture. Joints and nerves become stressed and begin to age more quickly. There may be lessening of overall energy and even a slight loss of height, with little pain or discomfort; Stage 2: There is a greater degree of disc decay, as the discs become narrower and bone deformations (bone spurs) appear. The changes in posture become more marked and spinal canal narrowing may also begin. Pain and discomfort are more evident and height may continue to decrease. More overall exhaustion but also a lowered ability to cope with stress; Stage 3: Posture is markedly affected. There is even greater disc thinning and also can moderate to severe nerve damage and scar tissue formation. In more advanced cases, further bone deformation occurs. Deterioration of the overall physical and/or mental condition of the patient and a profound loss of energy; and Stage 4: Severe damage to the spine. Discal thinning is at its maximum, or worse, completely gone. Postural imbalance is acute and motion and flexibility are extremely limited. Patients often suffer profound nerve damage, while permanent scar tissue forms and bones may begin to fuse. Severe pain and more advanced degrees of physical and/or mental deterioration are experienced. There is also an ongoing loss of height and energy levels. Stage 4 DDD is usually considered irreversible.

The present disclosure is further illustrated by the following examples that should not be construed as limiting.

### EXAMPLES

### Example 1: PCRX-201 Infection does not Affect Viability of NP Cells (Resazurin Reduction Assay)

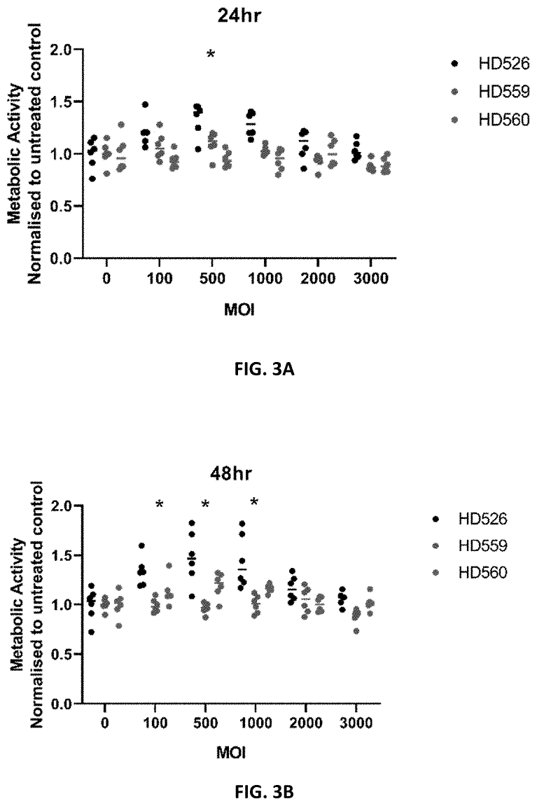

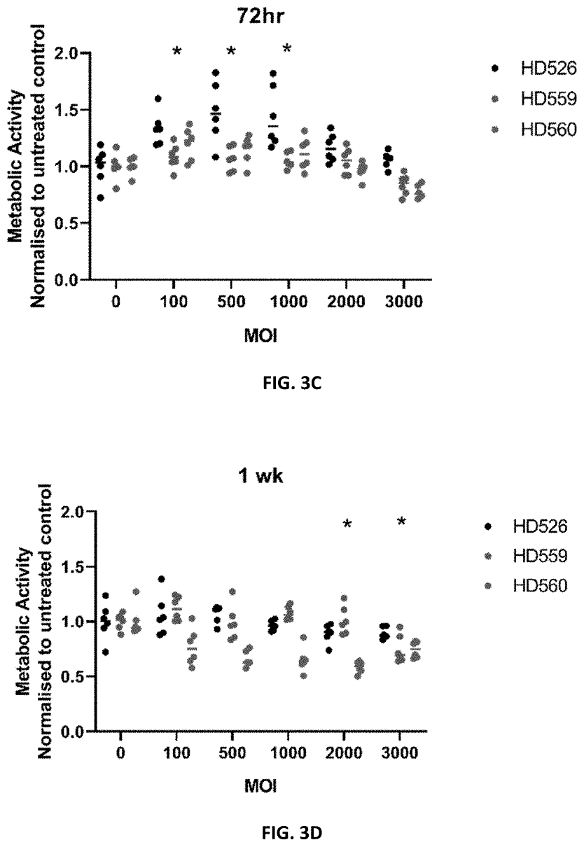

Human nucleus pulposus (NP) cells were isolated via collagenase digestion from surgical samples. Tissue from each surgical sample was also embedded to paraffin wax for histological grading utilizing consensus grading system for human IVD degeneration grading the NP region only, on a scale of 0-9 where 0-3 is non-degenerate, 4-6 moderate degenerate and 7-9 severely degenerate. NP cells derived from non-degenerate and degenerate discs were expanded in monolayer culture up to a maximum of passage 2 and were infected with PCRX-201 (also known as FX201) at 0, 100, 500, 1000, 2000 and 300 multiplicity of infection (MOI) and metabolic activity (as a proxy measure of viability) investigated using resazurin reduction assay at 24 hr, 48 hr, 72 hr and 1 wk. No loss of metabolic activity was observed up to 72 hrs following PCRX-201 infection even at the highest MOI of 3000, although a small decrease in metabolic activity was observed following 1 wk infection with MOI 2000 and 3000 (P<0.05) (FIG. 3).

### Example 2: PCRX-201 Infection does not Effect Viability of NP Cells (DNA Content)

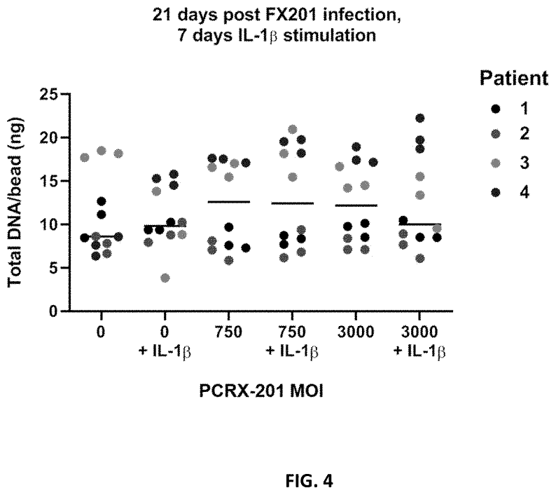

NP cells derived from degenerate human IVDs (n=6: Graded: 4, 5, 5, 6, 7 and 8), were infected in monolayer at an MOI of 0, 750 and 3000 with PCRX-201 for 48 hrs and then transferred to alginate bead cultures to mimic in vivo phenotype, induce re-differentiation and proliferation rates. Alginate beads were maintained in low glucose media at 5% O₂ to mimic the in vivo environment for up to 21 days+/−stimulation with a physiologically relevant concentration of IL-1β (100 μg/ml) for the final 7 days of culture. IL-1Ra production determined using ELISA, and viability assessed via DNA content using the Pico-green quantification in NP cells derived from 4 patients (Grades 5, 5, 7, 8). No decrease in DNA content was observed following 21 days of alginate culture post PCRX-201 infection (FIG. 4), suggesting no adverse effect on cell viability.

### Example 3: PCRX-201 can Induce IL-1Ra Gene and Protein Expression in Human IVD Cells Derived from Non-Degenerate and Degenerate Discs

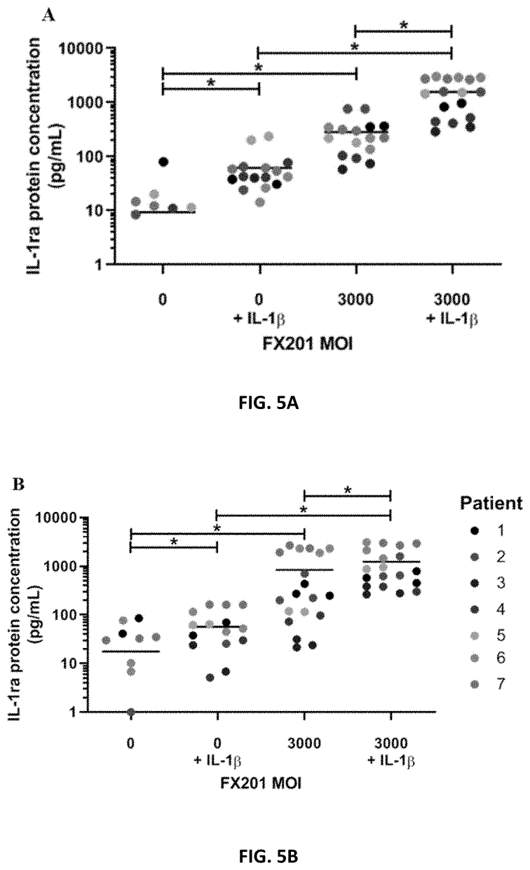

To investigate whether PCRX-201 could induce IL-1Ra protein in NP cells derived from degenerate discs, 7 patient samples (grades: 4, 4, 5, 5, 8, 9 and 9) were infected with PCRX-201 in monolayer culture at passage 2 at a MOI of 3000 for 48 hrs alone or in combination with additional 10 ng/ml IL-1β stimulation. Conditioned media collected and IL-1Ra production determined using ELISA 48 hrs and 120 hrs post infection with and without IL-1β stimulation. Stimulation of non-infected human NP cells in monolayer culture with IL-1p induced a significant increase in IL-1Ra production by NP cells following 48 hrs and 120 hrs (P<0.05) (FIGS. 5A and 5B). Infection with PCRX-201 in monolayer culture induced a large ˜100 fold increase in IL-1Ra protein secretion into the culture media following 48 and 120 hrs post infection (P<0.05) (FIGS. 5A and 5B), with production further enhanced following IL-1β stimulation. The results of the study described herein demonstrated that PCRX-201, has the ability to infect and increase the production of IL-1Ra in degenerate human NP cells in monolayer and 3D conditions in vitro.

### Example 4: Maintenance of PCRX-201 Over Prolonged Culture Periods in Conditions which Mimic the In Vivo Environment and Determine Release Profile of IL-1Ra

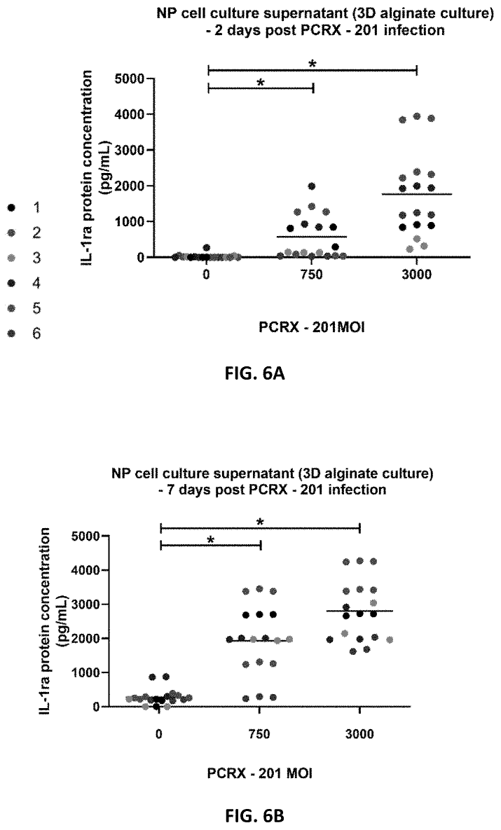

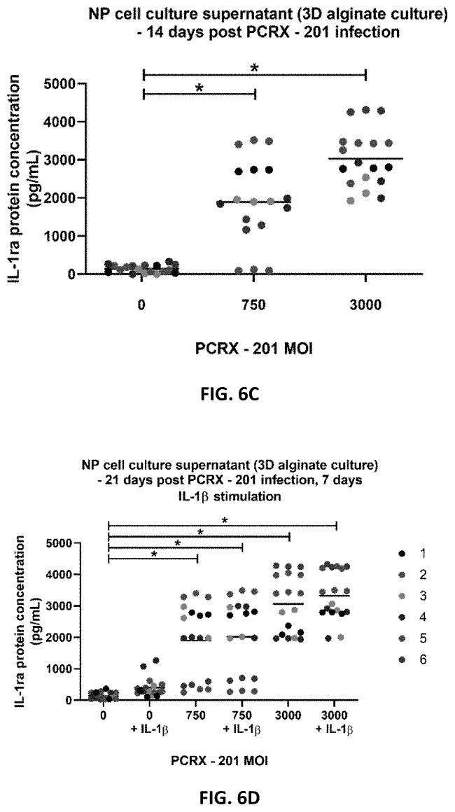

NP cells derived from degenerate human IVDs (n=6: Graded: 4, 5, 5, 6, 7 and 8), were infected in monolayer at an MOI of 0, 750 and 3000 with PCRX-201 for 48 hrs and then transferred to alginate bead cultures to mimic in vivo phenotype, induce re-differentiation and proliferation rates. Alginate beads were maintained in low glucose media at 5% O₂ to mimic the in vivo environment for up to 21 days+/−stimulation with a physiologically relevant concentration of IL-1β (100 μg/ml) for the final 7 days of culture. IL-1Ra production determined using ELISA. IL-1Ra protein secretion by NP cells infected with PCRX-201 showed a MOI dose dependent increase following 2, 7, 14 and 21 days in alginate culture with maintenance of prolonged IL-1Ra production over time in alginate culture with ˜2000 fold increase in IL-Ra production induced after 21 days in culture with MOI 750 and ˜3000 fold increase following MOI 3000 (P<0.05) (FIG. 6A-6D). Stimulation with IL-1 at a physiological concentration (100 μg/ml) did not induce a significant increase in IL-1Ra release (FIG. 6A-6D).

### Example 5: Long-Term Maintenance of IL-1Ra Production from Degenerate NP Cells Infected with PCRX-201

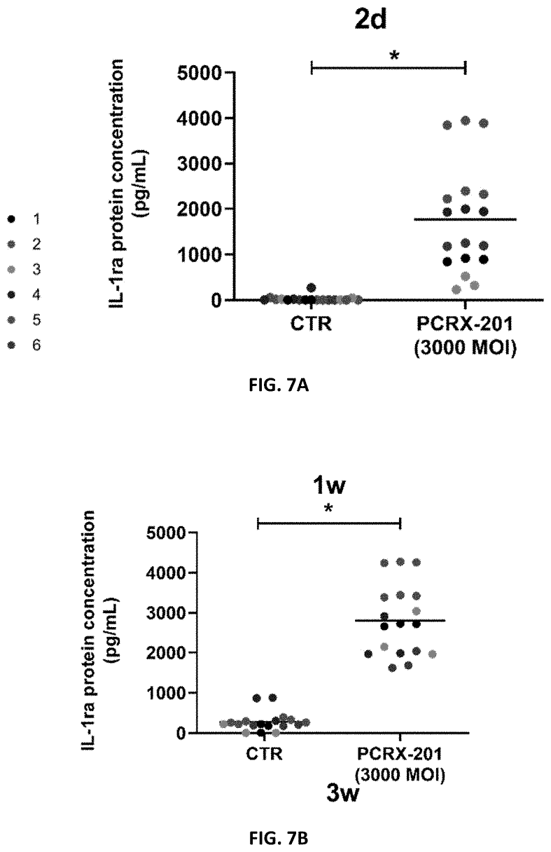

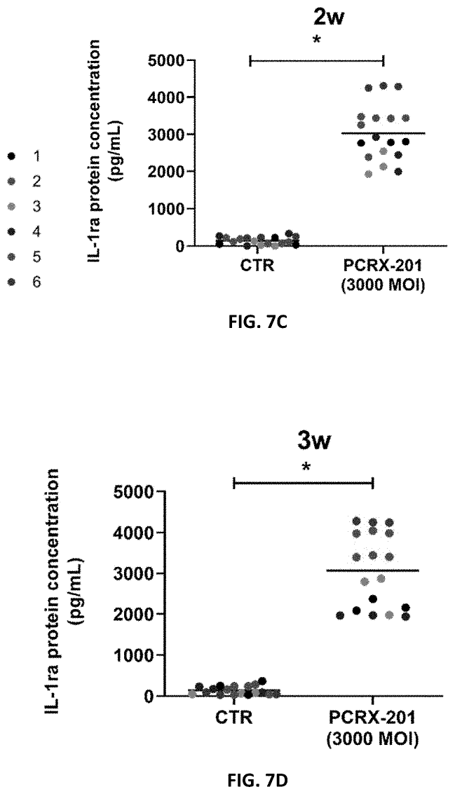

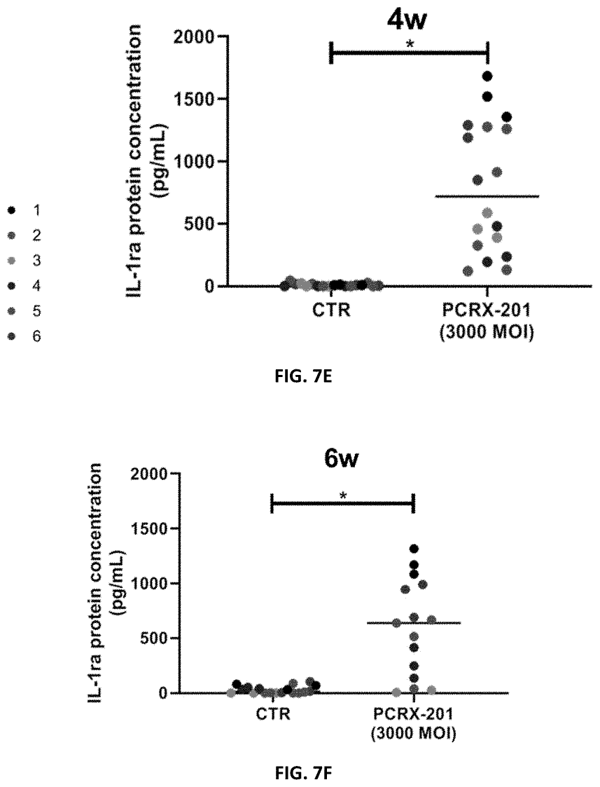

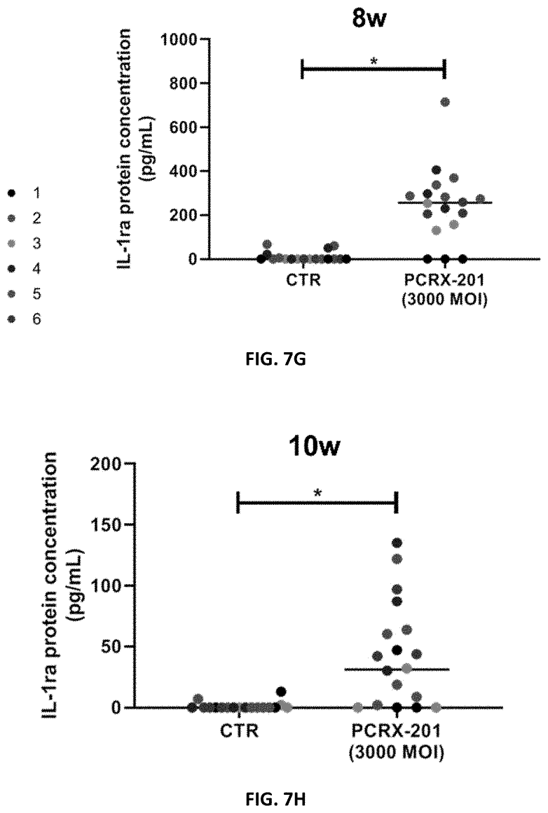

To investigate maintenance of IL-1Ra production from degenerate NP cells infected with PCRX-201, the cultures described above were maintained in low glucose DMEM media at 5% O₂ to mimic physiological conditions for up to 10 weeks. IL-1Ra release into culture media was quantified following 1, 2, 3, 4, 6, 8 and 10 wks post infection. IL-1 Ra production from cells infected with PCRX-201 was significantly enhanced even following 10 weeks in culture (P<0.05) (FIG. 7), although following 8 weeks in culture alginate culture integrity was decreased with loss of cells from alginate cultures which coincided with lower IL-1Ra content in conditioned media. These results demonstrate that the production of IL-1Ra protein induced by infection of PCRX-201 in NP cells isolated from human degenerate discs is maintained to high levels in a 3D culture environment which mimics native physiological conditions of low glucose and 5% O₂ for at least 10 wks.

### Example 6: Efficacy of IL-1Ra Induction by PCRX-201 in Human IVD Cells on Catabolic Features of Disc Degeneration and Release of Pain Inducing Factors

NP cells derived from degenerate human NP tissue (n=6; Graded: 4, 5, 5, 6, 7 and 8), were infected in monolayer at MOI of 0, 750 and 3000 and transferred to alginate bead cultures. Cultures were maintained in low glucose media at 5% O₂ to mimic the in vivo environment for 14 days prior to stimulation+/−100 μg/ml IL-1 for a further week. To determine the ability of PCRX-201 to inhibit expression of angiogenic and neurotrophic factors: NGF, VEGF, cytokines: IL-1, IL-6, and IL-8; and matrix degrading enzymes: MMP 3 and ADAMTS 4 were determined investigating release of protein into conditioned media. Gene expression of NGF, was also investigated.

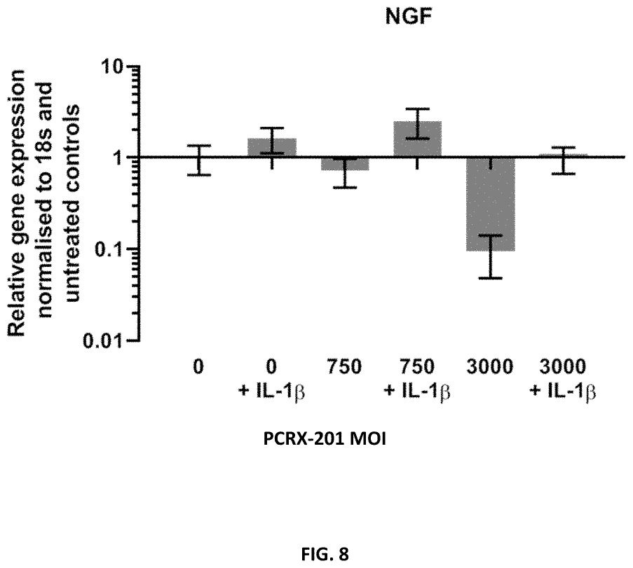

NGF protein expression was only detected at low levels within one patient sample with other patients being below the detectable limit of the ELISA, thus gene expression analysis was −60-tilized to investigate potential influence of PCRX-201 on NGF production. NGF mRNA expression was increased slightly by 100 μg/ml IL-1β stimulation, whilst infection with PCRX-201 induced a dose dependent decrease in NGF mRNA expression (FIG. 8).

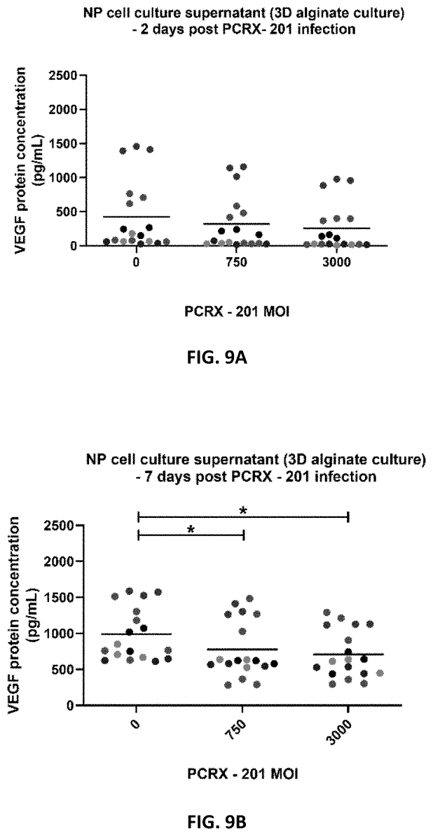

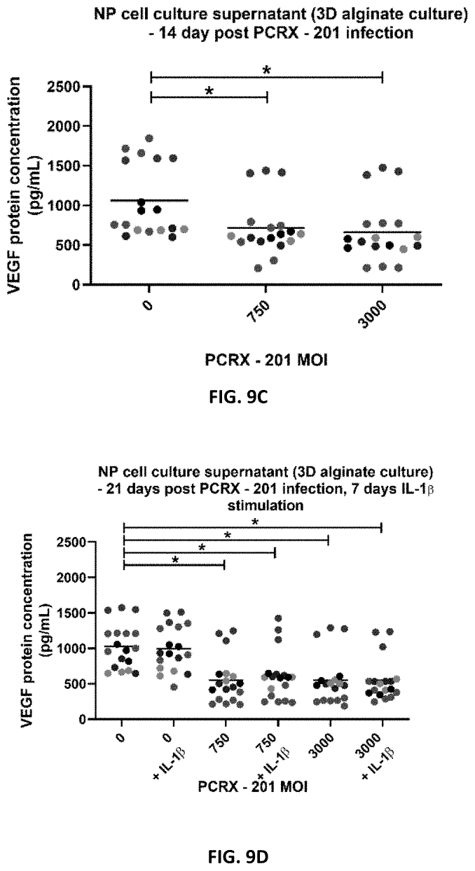

VEGF production was significantly decreased in cells infected with PCRX-201 after 7, 14 and 21 days in culture, whilst 100 μg/ml IL-1β did not induce an increase in VEGF (FIG. 9A-9D).

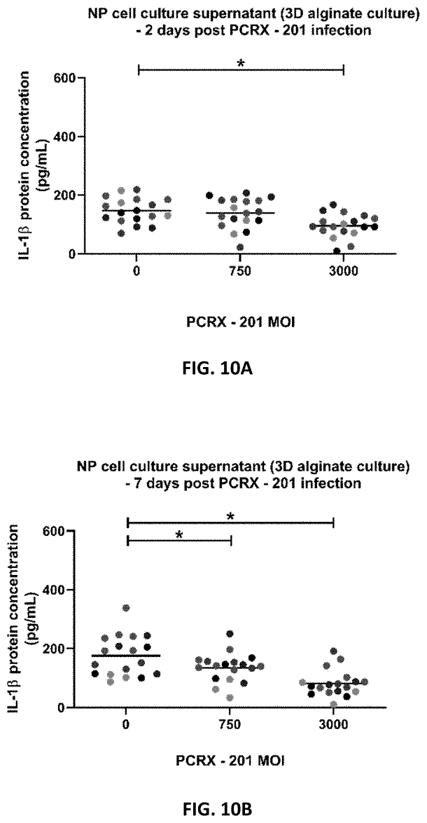

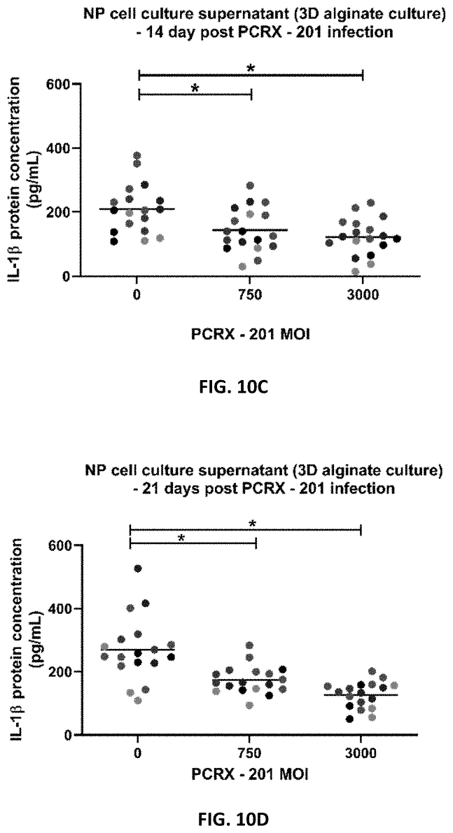

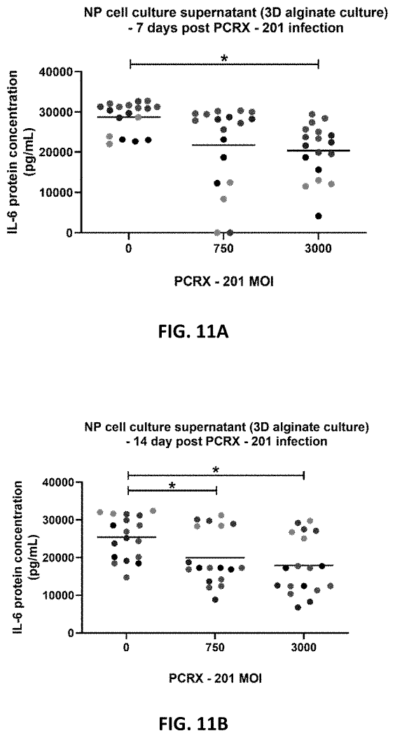

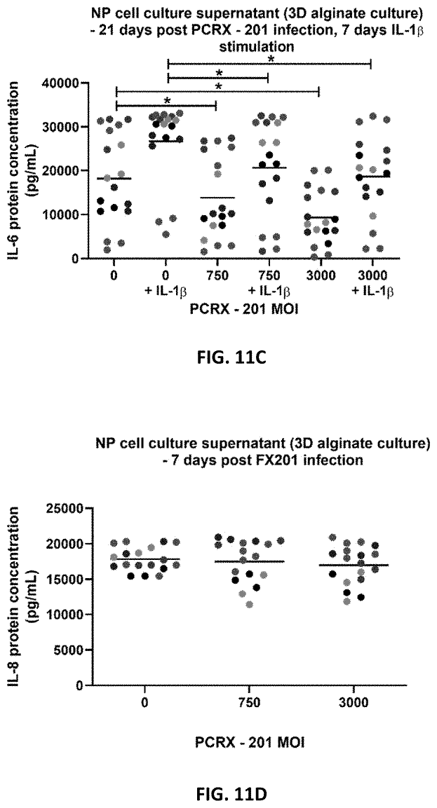

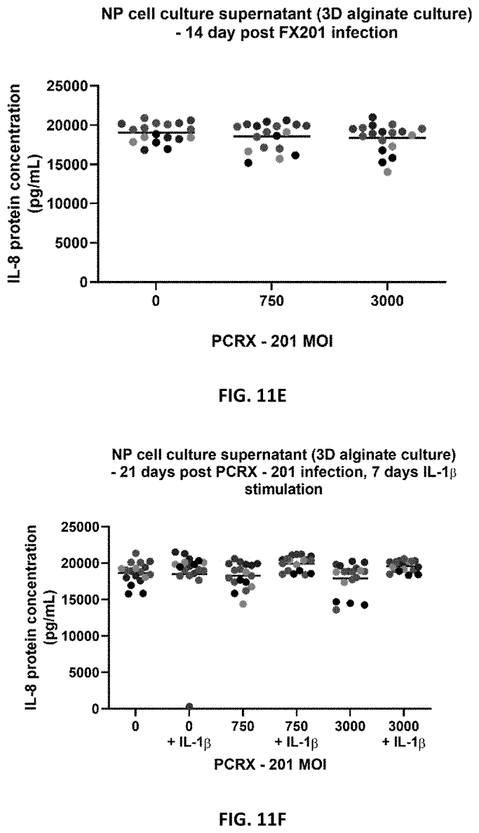

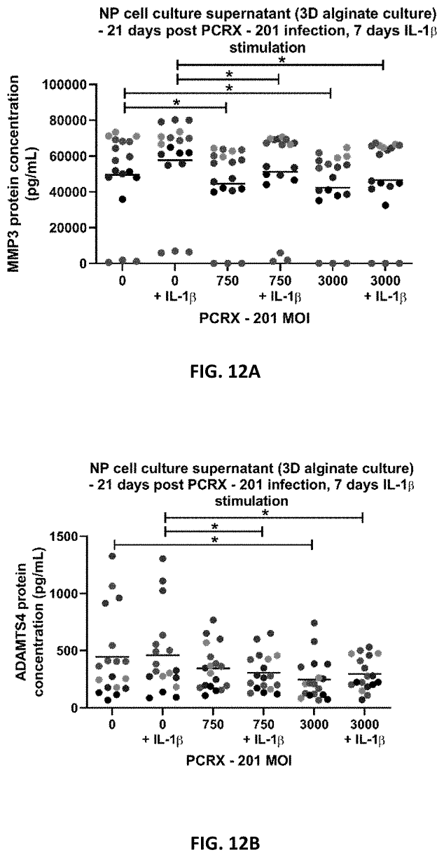

IL-1β protein release was significantly decreased in a dose dependent manner following PCRX-201 infection at 2, 7, 14 and 21 days in culture (P<0.05) (FIGS. 10A-10D). IL-6 and IL-8 protein was not detected in many patient samples in culture supernatant following 2 days of culture. IL-6 was significantly decreased in a dose dependent manner following PCRX-201 infection at 7, 14 and 21 days in culture, stimulation with IL-1β increased IL-6 production in all treatment groups (P<0.05) (FIG. 11A-11C). In contrast IL-8 production was not affected by either IL-1β stimulation nor infection with PCRX-201 (FIG. 12A-12C).

Production of matrix degrading enzymes MMP 3 and ADAMTS 4 were investigated in NP cells derived from degenerate discs infected with PCRX-201 at MOI 750 and 3000 and cultured in alginate for 14 days to enable re-differentiation prior to stimulation with 100 μg/ml IL-1β for a further week. MMP 3 and ADAMTS 4 were significantly down regulated following PCRX-201 at both 750 and 3000 MOIs (P<0.05), whilst IL-1β stimulation induced a small but not significant increase in MMP 3 and ADAMTS 4 production in non-infected cells only (FIGS. 12A and 12B).

These results demonstrate that infection of NP cells derived from degenerate disc with PCRX-201 decreases the production of key catabolic, angiogenic and neurotrophic factors in the cells infected with PCRX-201.

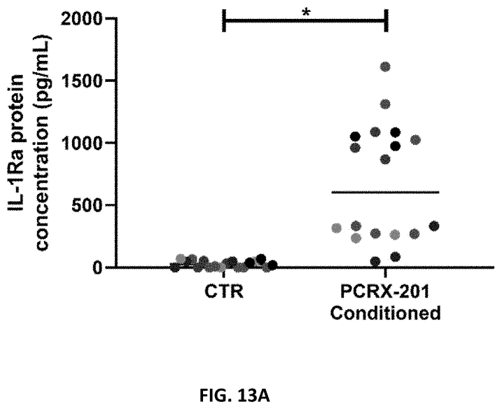

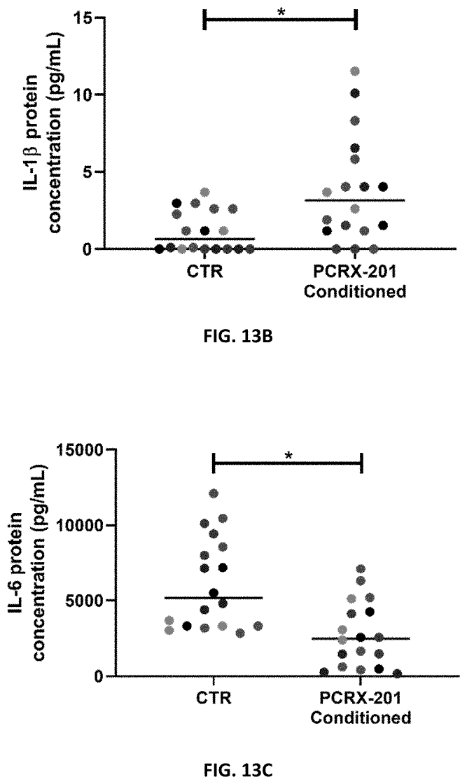

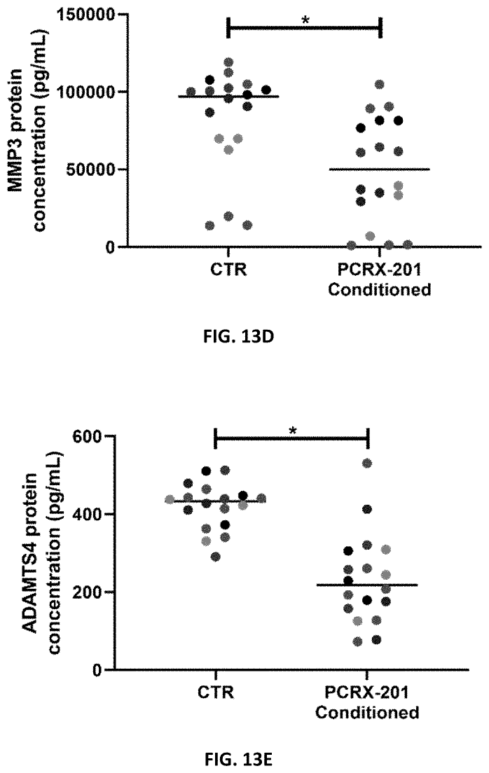

To determine whether IL-1Ra released from PCRX-201 infected NP cells could also act in a paracrine fashion, and thus deliver potentially a further removed effect to the disc beyond those cells directly infected with PCRX-201. Conditioned media was collected from patient matched human NP cells infected with PCXR-201 and utilized to treat non-infected control cells for a period of 72 hrs in low glucose media at 5% O₂. The protein production of IL-1Ra (FIG. 13A), IL-1β (FIG. 13B), IL-6 (FIG. 13C), MMP 3 (FIG. 13D) and ADAMTS 4 (FIG. 13E) by untreated non-infected control cells and those stimulated with the conditioned media from PCRX-201 patient matched cells was determined using ELISA. Conditioned media from PCRX-201 cells significantly decreased the protein production of IL-6, MMP 3 and ADAMTs 4 in non-infected cells in a paracrine fashion (P<0.05), whilst a small increase in IL-1β was observed very low levels were seen compared to the enhanced concentration of IL-1Ra.

These results demonstrate that infection of NP cells derived from degenerate disc with PCRX-201 decreases the production of key catabolic, angiogenic and neurotrophic factors in the cells infected with PCRX-201 and local cells in a paracrine manner.

### Example 7: The Ability of PCRX-201 to Induce IL-1Ra Production in Human IVD Tissue Following Direct Injection Using an Explant Model

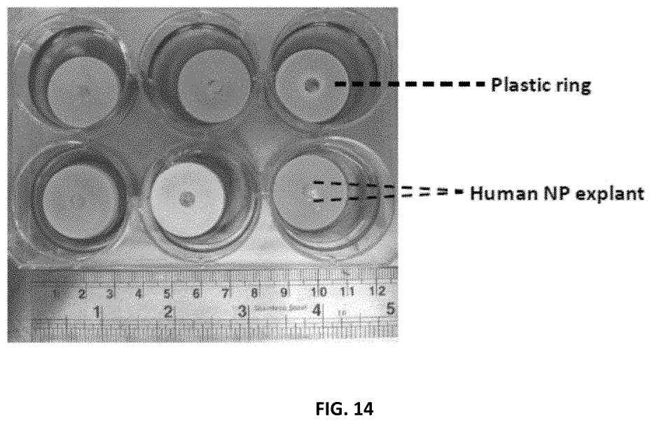

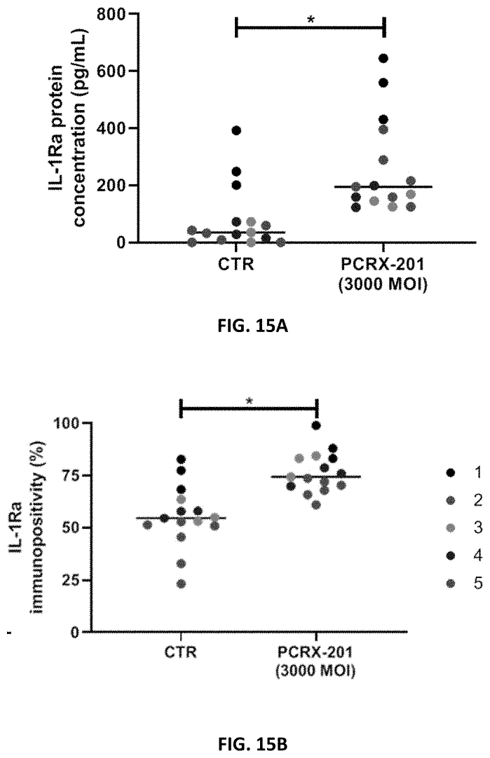

To determine whether PCRX-201 could be utilized to deliver IL-1Ra directly within the IVD as opposed to ex vivo gene delivery PCRX-201 was injected directly to human IVD tissue explants cultured within a semi-constrained culture system which maintains NP tissue integrity and phenotype (FIG. 14). Five patient samples (Histological grades: 4, 5, 6, 7 and 8) were utilized for this study. Tissue explants were divided into three treatment groups, non-injected controls, PCRX-201 injected (50 μl per explant). Infectivity rates were calculated based on a cell density of disc tissue of 4×106 cells/mm3 and a tissue explant area of 98.2 mm3 (5 mm diameter core, 5 mm high) to generate a MOI of 3000. All treatments were performed in triplicate for up to 2 weeks in low glucose, 5% O₂ culture. Cell culture supernatant was harvested to investigate release of IL-1Ra into the media via ELISA, in addition tissue explants were processed to wax and immunohistochemistry utilized to investigate the expression of IL-1Ra in the tissue explants. A significant increase in both IL-1Ra protein release from tissue explants and the number of cells within the NP tissue immunopositive for IL-1Ra was seen 2 weeks following injection of PCRX-201 into NP tissue explants (FIGS. 15A-15B).

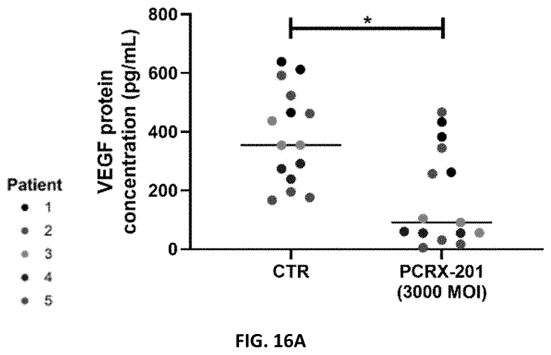

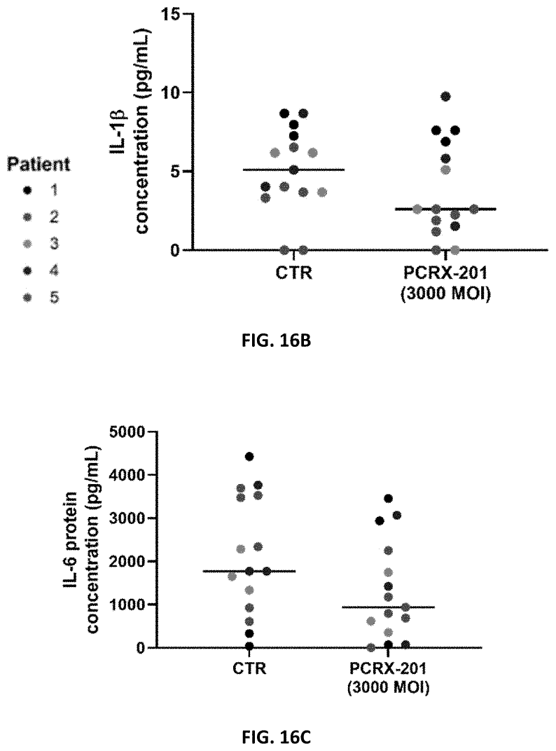

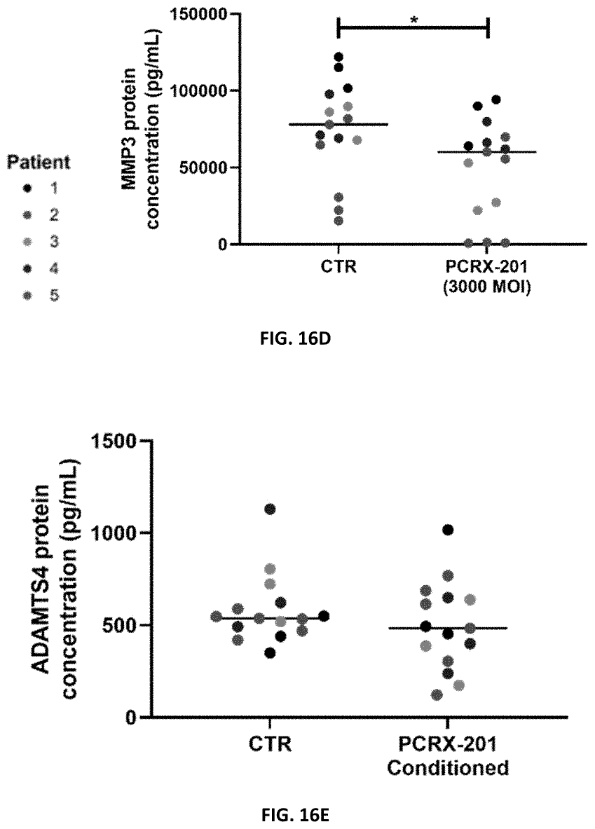

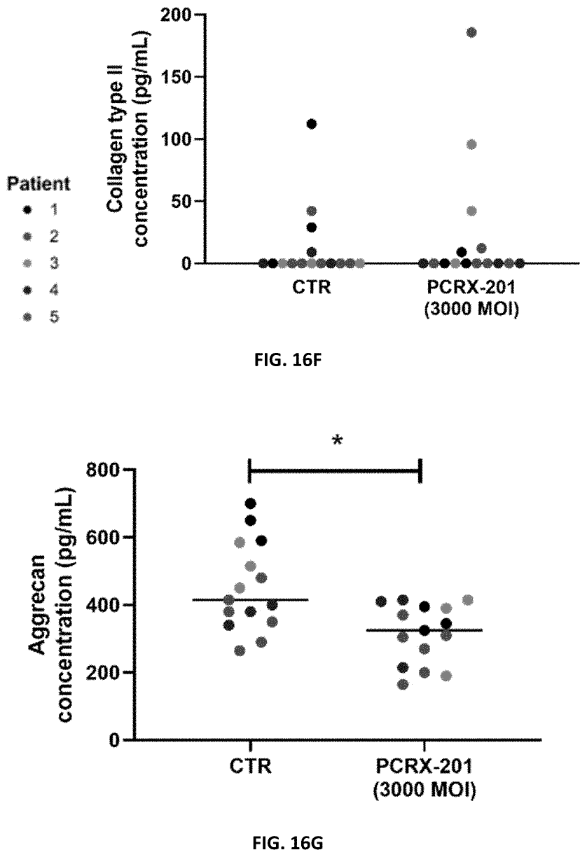

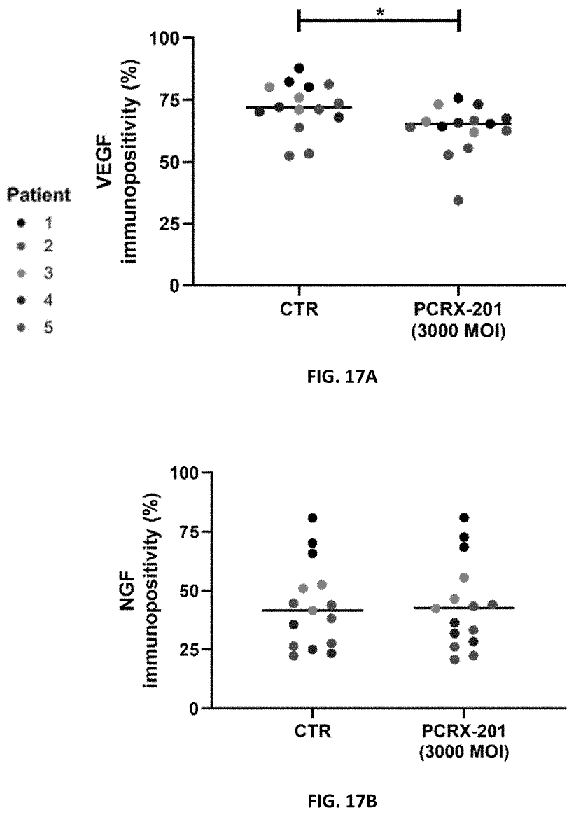

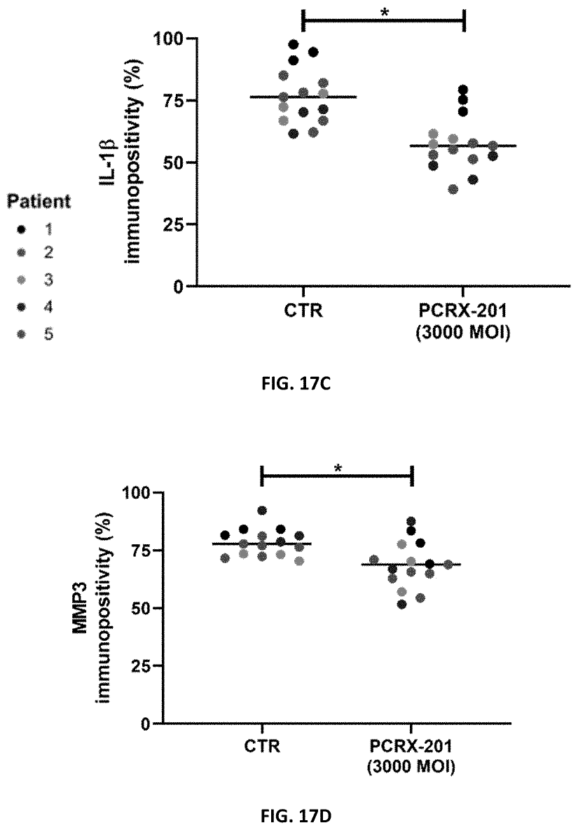

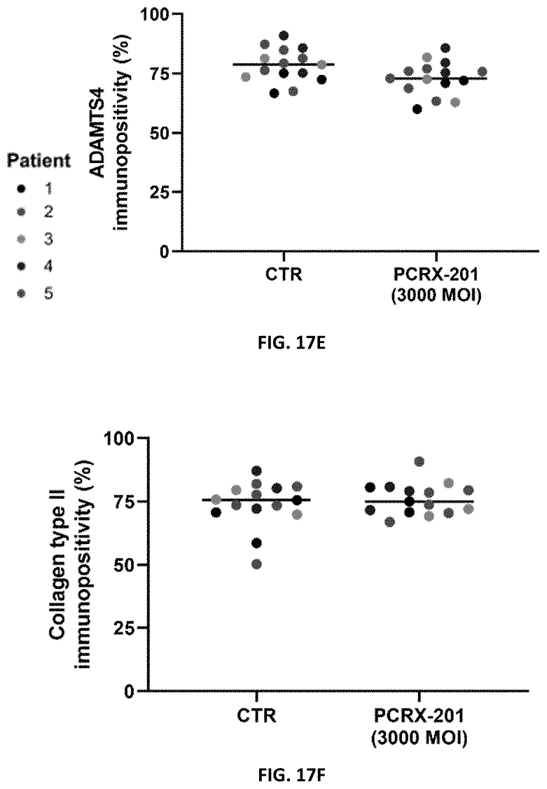

Furthermore, these disc explants were utilized to investigate whether direct injection of PCRX-201 into native NP tissue could promote anabolism and inhibit the catabolic processes in the degenerate discs. Cell culture supernatant was analyzed using ELISA for VEGF, IL-1β, IL-6, MMP 3, ADAMTs 4, Collagen type II and Aggrecan and immunohistochemistry performed on paraffin embedded tissue explants to determine the percentage of cells within each explant which were immunopositivity for VEGF, NGF, IL-1β, MMP 3, ADAMTs 4, Collagen type II and Aggrecan. The injection of human NP tissue explants derived from degenerate discs with PCRX-201 significantly decreased the concentration of VEGF (FIG. 16A), and MMP3 (FIG. 16D) a decrease in IL-1β (FIG. 16B) and IL-6 (FIG. 16C) was also observed yet this failed to reach significance. ADAMTs 4 protein concentrations released into culture media were not altered 2 weeks after injection (FIG. 16E). Very limited collagen type II was observed in conditioned media (FIG. 16F), whilst aggrecan was released into the media which was significantly decreased in those tissue explants which were injected with PCRX-201, potentially due to reduced degradation (FIG. 16G). Immunohistochemical analysis confirmed these results with a significant decrease in number of immunopositive cells for VEGF (FIG. 17A), IL-1β (FIG. 17C) and MMP 3 (FIG. 17D). A small but non-significant decrease in percentage immunopositive cells was also seen for ADAMTS 4 (FIG. 17E). Whilst the number of immunopositive cells for NGF (FIG. 17B) and Collagen type II (FIG. 17F) were not affected 2 weeks post injection.

These data provide evidence that PCRX-201 can induce increased IL-1Ra production within native tissue via direct injection, and that this induces reduced catabolism and decreased degradation of aggrecan.

### Conclusions:

Together these data performed using cells and tissues isolated from surgically removed human IVD tissue has demonstrated PCRX-201 can successfully increase active IL-1Ra protein production by NP cells derived from degenerate discs. The expression of IL-1Ra protein by infected cells could be maintained long term (up to the 10 weeks investigated) in 3D culture system which mimics the IVD environment in terms of glucose, O₂, cellular phenotype and proliferative capacity. The IL-1Ra induced resulted in down regulation in infected cells and in a paracrine fashion cells via treatment with conditioned media, of key catabolic factors (degrading enzymes and catabolic and inflammatory cytokines), angiogenic and neurotrophic factors (VEGF and NGF). Furthermore, direct injection of PCRX-201 into native disc tissue in an ex vivo culture system induced increased IL-1Ra production and expression in injected discs, with a concordant decrease in catabolism.

1. A pharmaceutical composition for treatment of degenerative disc disease (DDD) or a condition associated with DDD, in a subject in need thereof, comprising an adenoviral-based biological delivery and expression system comprising a nucleic acid encoding a interleukin-1 receptor antagonist (IL-1Ra) protein.

2.-21. (canceled)

22. A method of expressing IL-IRA in cells of one or more intervertebral discs of a subject suffering from degenerative disc disease (DDD) or a condition associated with DDD the method comprising:
a) infecting cells of one or more intervertebral discs of the subject in need thereof with the pharmaceutical composition of claim 1; and
b) expressing IL-1Ra in the cells of the one or more intervertebral discs.

23. The method of claim 22, wherein the adenoviral-based biological delivery and expression system further comprises a nucleic acid encoding a protein in addition to interleukin-1 receptor antagonist (IL-1Ra) protein.

24. The method of claim 22, wherein the cells of the one or more intervertebral discs are infected once with the adenoviral-based biological delivery and expression system.

25. The method of claim 22, wherein the cells of the one or more intervertebral discs are infected two or more times with the adenoviral-based biological delivery and expression system.

26. The method of claim 25, wherein when the cells of the one or more intervertebral discs are infected two or more times with an adenoviral-based biological delivery and expression system, each infection comprises a different number of genome copies of the adenoviral-based vector.

27. The method of claim 25, wherein, when the cells of the one or more intervertebral discs are infected two or more times with an adenoviral-based biological delivery and expression system, each infection comprises the same number of genome copies of the adenoviral-based vector.

28. The method of claim 25, wherein, when the cells of the one or more intervertebral discs are infected two or more times with an adenoviral-based biological delivery and expression system, each infection is done in the same intervertebral disc of the subject.

29. The method of claim 25, wherein when the cells of the one or more intervertebral discs are infected two or more times with an adenoviral-based biological delivery and expression system, every second and subsequent infection is done in an intervertebral disc of the subject that is different than the intervertebral disc in which the previous infection was done.

30. The method of claim 22, wherein the infecting of the cells of the one or more intervertebral discs comprises injecting the pharmaceutical composition into the cartilaginous endplates (CEP) region, the highly organized annulus fibrosus (AF) region or the central gelatinous nucleus pulposus (NP) region (nucleus pulposus (NP) cells) or a combination thereof, of the one or more intervertebral discs.

31. The method of claim 30, wherein the infecting of the cells of the one or more intervertebral discs comprises injecting the pharmaceutical composition into the nucleus pulposus (NP) region of the one or more intervertebral discs.

32. The method of claim 22, wherein the method treats the degenerative disc disease (DDD) and/or the condition associated with DDD in the subject.

33. The method of claim 22, further comprises the step of:
c) monitoring the treatment or progress of DDD or the condition associated with DDD in the degenerated intervertebral discs of the subject following the expression of the IL-1Ra in (b).

34. The method of claim 33, wherein the monitoring of the treatment or progress of DDD or the condition associated with DDD, is done by determining scores from patient-reported pain using visual analog scale (VAS) and/or function measurements using Oswestry disability index (ODI).

35. (canceled)

36. The method of claim 34, wherein a VAS score or ODI of the subject is lower post-infection of the cells of one or more intervertebral discs of the subject with the pharmaceutical composition.

37-38. (canceled)

39. The method of claim 33, wherein the monitoring of the treatment or progress of DDD or the condition associated with DDD comprises determining the level of a marker in the subject selected from the group consisting of: NGF, NT-3, VEGF, Substance P, cytokines, aggrecan, collagen type II, and a combination thereof, wherein the cytokine is selected from the group consisting of IL-1β, IL-6, IL-8, TNF α, MMP 3, MMP 13, ADAMTS 4, and combinations thereof.

40. (canceled)

41. The method of claim 39, wherein one or more of the following occurs:
(a) a decrease or no change in level of one or more of: NGF, NT-3, VEGF, Substance P, IL-1β, IL-6, IL-8, TNF α, MMP 3, MMP 13, or ADAMTS 4, or a combination thereof;
(b) an increase in the level of aggrecan or collagen type II or a combination thereof; or
(c) both,
in the one or more intervertebral discs post-infection of the cells of one or more intervertebral discs of the subject with the pharmaceutical composition.

42. The method of claim 41, wherein level of aggrecan increases in the one or more intervertebral discs post-infection of the cells of one or more intervertebral discs of the subject with the pharmaceutical composition.

43. The method of claim 33, wherein the monitoring of the treatment or progress of DDD or the condition associated with DDD, is done by determining change in a score based on histopathology scoring system for human intervertebral disc degeneration, and wherein the histopathology scoring system improves for the one or more intervertebral discs post-infection of the cells of one or more intervertebral discs of the subject with the pharmaceutical composition.

44. (canceled)

45. The method of claim 33, further comprises the steps of:
(d) continuing to administer the same amount of the adenoviral-based biological delivery and expression system to the cells of the one or more intervertebral discs of (a), if monitoring of (c) shows that the degenerative disc disease in the intervertebral disc of the subject is not managed or treated; or
(e) further adjusting the amount of the adenoviral-based biological delivery and expression system and administering to the cells of one or more intervertebral discs of the subject in need thereof, of (a), if monitoring of (c) shows that the degenerative disc disease in the intervertebral disc of the subject has progressed.

46. The method of claim 22, wherein the method further comprises administration of a corticosteroid and/or a local anesthetic into the intervertebral disc of the subject.

47. (canceled)

48. (canceled)

49. The method of claim 22, wherein the method further comprises administration of a fluid into the intervertebral disc of the subject after the adenoviral-based biological delivery and expression system, optionally wherein the amount of fluid is 25-1000 μl.

50. The method of claim 49, wherein the fluid is saline.

51. The method of claim 50, wherein the further administration comprises intradiscal injection of the fluid.

52. The method of claim 22, wherein the subject does not have Facet Joint Syndrome (FJS).

53. The method of claim 22, wherein the subject has Facet Joint Syndrome (FJS).

54. The method of claim 22, wherein the method comprises intradiscal injection of the pharmaceutical composition.

55. The method of claim 54, wherein the intradiscal injection is to the central gelatinous nucleus pulposus (NP) region.

56. The method of claim 22, wherein the method does not comprises intra-tendinous, intra-muscular, intra-articular, or sub-acromial injection of the pharmaceutical composition.

57. The method of claim 22, wherein the concentration of the adenoviral-based biological delivery and expression system in the pharmaceutical formulation is about, 1×10⁸ to 5×10¹¹ VP/ml.

58. The method of claim 22, wherein the concentration of the adenoviral-based biological delivery and expression system in the pharmaceutical formulation is about, 1×10⁸ to 5×10¹¹ GC/ml.

59. The method of claim 22, wherein a single dose of the pharmaceutical composition administered to an intravertebral disc is an amount that is about 0.1 ml to 5 ml.

60. The method of claim 22, wherein the infected cells comprise NP cells.

61. (canceled)

62. The method of claim 22, wherein the nucleic acid further comprises left and right inverted terminal repeats, an adenoviral packaging signal and non-viral, and non-coding stuffer nucleic acid sequences.

63. The method of claim 62, wherein the nucleic acid further comprises an inflammation-sensitive promoter located upstream of the reading frame of the nucleic acid sequence encoding the IL-1Ra protein, such that expression of the IL-1Ra gene is regulated by the inflammation-sensitive promoter.

64. The method of claim 62, wherein the nucleic acid further comprises an NF-kB inducible promoter located upstream of the reading frame of the nucleic acid sequence encoding the IL-1Ra protein, such that expression of the IL-1Ra gene is regulated by the NF-kB inducible promoter.

65. The method of claim 22, wherein the nucleic acid consists of a nucleic acid sequence that is at least 90% identical to the nucleic acid sequence of SEQ ID NO: 7.

66. The method of claim 22, wherein the nucleic acid consists of the nucleic acid sequence of SEQ ID NO: 7.

67. The method of claim 22, wherein the condition associated with DDD is lower back pain, decreased back muscle tone, reduced flexibility of the back, blood clot or a combination thereof.

68. The method of claim 22, wherein the cells of one or more intervertebral discs of the subject in need thereof, express IL-1Ra for a period of at least 2 weeks following step a).


SEQUENCE LISTING (7 sequences)

SEQ ID NO: 1 - DNA, 535 bp, synthetic construct
  misc_feature 1..535: note = murine Il-1Ra
  source 1..535: mol_type = other DNA
atggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaat

SEQ ID NO: 2 - DNA, 29612 bp, synthetic construct
  misc_feature 1..29612: note = helper-dependent adenoviral vector-murine Il-1Ra gene
  source 1..29612: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgctcctctagactcgaggaattcggtaccccgggttcgaaatcgataagcttggatcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaggatctgcgatcgctgaattctggggactttccactggggactttccactggggactttccactggggactttccactggggactttccactcctgcagcagtggatattcccagaaaactttttggatgcagttggggatttcctctttactggatgtggacaatatcctcctattattcacaggaagcaatccctcctataaaagggcctcagaggaagtagtgttcagctgttcttggctgacttcacatcaaagctcctatactgacctgagacagagccatgaattccgtttttttttttttttttggaaatatgagggtttttccgcttctgacagtggaacggaatgacagcagcacaggctggtgaatgactactttctttataagcaaccaccttgagcctgaaatggcagtcgctagtctctattgccttgctgtggcctcgggatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttagatccggagagctcccaacgcgttgggtcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 3 - DNA, 29273 bp, synthetic construct
  misc_feature 1..29273: note = helper-dependent adenoviral vector-equine Il-1Ragene
  source 1..29273: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgctcctctagactcgaggaattcggtaccccgggttcgaaatcgataagcttggatccggagagctcgataacatttaaatggatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgagctaggatcccgcggccgcctactggtcctcctggaggtagaacttggtgaccatgaaggactctttgggcttgttggtgaggctgacgggccggtctgcctcctgcgccgtgcagaggaaccagccagggcaggcggcagactcgaagctggtggtggggccactgtttgagcggatgaaggtgaagcgcttgttctcctccttgttcttgctcaggtcagtgatgttaactgcctccaattggaacctaatctcatcaccagacttgacacaggccaggcacagcttcctcccatggagtcccaggaatagagcatcaggctcaatgggcaccacatctatcttctcttgtaatttagtatttgattcttgcaagtatccagcaactagttggttattcctcatgtagaaggtcttctggttaacatcccagattctgaaggcttgcatcttgcagggtctcttccccaaagggtggcaggctgtctctgagtagagcaagaaaaggaggagagagattaggtgtctgacagaacgcctgcggatttccataccggtggatccatggctctgtctcaggtcagtataggagctttgatgtgaagtcagccaagaacagctgaacactacttcctctgaggcccttttataggagggattgcttcctgtgaataataggaggatattgtccacatccagtaaagaggaaatccccaactgcatccaaaaagttttctgggaatatccactgctgcaggagtggaaagtccccagtggaaagtccccagtggaaagtccccagtggaaagtccccagtggaaagtccccagaatttcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 4 - DNA, 534 bp, synthetic construct
  misc_feature 1..534: note = codon optimized human IL-1Ra
  source 1..534: mol_type = other DNA
atggaaatctgcagaggcctgcggagccacctgattaccctgctgctgttcctgttccacagcgagacaatctgccggcccagcggcagaaagtccagcaagatgcaggccttccggatctgggacgtgaaccagaaaaccttctacctgcggaacaatcagctggtggccggctacctgcagggccccaatgtgaacctggaagagaagatcgacgtggtgcccatcgagccccacgccctgtttctgggaatccacggcggcaagatgtgcctgagctgcgtgaagtccggcgacgagacacggctgcagctggaagccgtgaacatcaccgacctgagcgagaaccggaagcaggacaagagattcgccttcatcagaagcgacagcggccccaccaccagctttgagtctgccgcttgccctggctggttcctgtgcactgccatggaagccgaccagcccgtgtccctgaccaacatgcctgacgagggcgtgatggtcaccaagttctattttcaagaggacgagtga

SEQ ID NO: 5 - DNA, 534 bp, synthetic construct
  misc_feature 1..534: note = original human IL-1Ra
  source 1..534: mol_type = other DNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 6 - protein, 177 aa, synthetic construct
  REGION 1..177: note = human IL-1Ra protein
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 7 - DNA, 29260 bp, synthetic construct
  misc_feature 1..29260: note = helper-dependent adenoviral vector-human Il-1Ragene
  source 1..29260: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaagtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagaatgcgccacttcactccagcctgggaaacagagcaagactctgtctctgagctgagatsgcrccacttcactccagcctgggaaacagagcaagactctgtctcaaaaaaaacaaaacaaacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactggggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtsgctcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcytggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactccgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagcttatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagattgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgagtatatatacatgtatatatactgtgtatatatacatgtatatatactgagtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgcgcatgctcctctagactcgaggaattcggtaccccgggttcgaaatcgataagcttggatccggagagctcgttaacatttaaatggatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgagatccgcgcggccgctcactcgtcctcttgaaaatagaacttggtgaccatcacgccctcgtcaggcatgttggtcagggacacgggctggtcggcttccatggcagtgcacaggaaccagccagggcaagcggcagactcaaagctggtggtggggccgctgtcgcttctgatgaaggcgaatctcttgtcctgcttccggttctcgctcaggtcggtgatgttcacggcttccagctgcagccgtgtctcgtcgccggacttcacgcagctcaggcacatcttgccgccgtggattcccagaaacagggcgtggggctcgatgggcaccacgtcgatcttctcttccaggttcacattggggccctgcaggtagccggccaccagctgattgttccgcaggtagaaggttttctggttcacgtcccagatccggaaggcctgcatcttgctggactttctgccgctgggccggcagattgtctcgctgtggaacaggaacagcagcagggtaatcaggtggctccgcaggcctctgcagatttccataccggtggatcatggctctgtctcaggtcagtataggagctttgatgtgaagtcagccaagaacagctgaacactacttctgctgaggcccttttataggagggattgcttcctgtgaataataggaggatattgtccacatccagtaaagaggaaatccccaactgcatccaaaaagttttctgggaatatccactgctgcaggagtggaaagtccccagtggaaagtccccagtggaaagtccccagtggaaagtccccagtggaaagtccccagaatttcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg